(19) United States
(12) Patent Application Publication
(10) Pub. No.: US 2024/0016788 A1
(43) Pub. Date: Jan. 18, 2024

(54) PHARMACEUTICAL COMPOSITION FOR PREVENTING OR TREATING DIABETIC AND POSTMENOPAUSAL OSTEOPOROSIS, CONTAINING ADIPORON

(71) Applicant: PnP Biopharm Co., Ltd., SEOUL (KR)

(72) Inventor: Cheol-Whee PARK, Seoul (KR)

(21) Appl. No.: 18/033,550

(22) PCT Filed: Nov. 12, 2021
(86) PCT No.: PCT/KR2021/016527

§ 371 Date: Apr. 24, 2023

(30) Foreign Application Priority Data
Nov. 13, 2020 (KR) 10-2020-0151788
Nov. 9, 2021 (KR) 10-2021-0153090

(51) Int. Cl. A61K 31/452; A61P 19/10

(52) U.S. Cl. CPC A61K 31/452; A61P 19/10

(57) ABSTRACT

The present invention relates to a pharmaceutical composition for preventing or treating diabetic and postmenopausal osteoporosis comprising adiporon and/or a method for treating diabetic and postmenopausal osteoporosis using the same. The composition of the present invention controls bone-related factors by activating the adiponectin-receptor 1-AMPK-Nrf2 signaling system and the adiponectin-receptor 2/PPARα-PGC-1α signaling system in bone tissue, improves lipotoxicity and can be usefully used as a therapeutic agent for diabetic and postmenopausal osteoporosis through growth plate promoting effects and chondrocyte proliferation effects in growth plates.

### TECHNICAL FIELD

The present invention relates to a pharmaceutical composition for preventing or treating diabetic and postmenopausal osteoporosis containing adiporon.

### BACKGROUND ART

The prevalence of diabetes in South Korea is 14.4% (diabetes society, 2016, population aged 30 years or older), and about 25% of diabetic patients have adequate diabetes control (glycated hemoglobin 6.5% or less). This low rate of diabetes control showed a 6.4-fold increase in the incidence of osteoporosis in type 1 diabetes and a 2.2-fold increase in type 2 diabetes and the risk of fracture increases 6.3 times in type 1 diabetes and 1.7 times in type 2 diabetes, compared to the non-diabetic group. Osteoporosis is a disease in which the skeleton is reduced due to a decrease in the quantity and quality of bones, and fracture is easily lost even with light shock. diabetic osteoporosis in type 1 diabetic patients results in a decrease in bone density due to a decrease in the function of osteoblasts that make bones due to the secretion of hormones from fat due to insulin deficiency and metabolic abnormalities, and in type 2 diabetic patients diabetic osteoporosis in type 1 diabetic patients results in a decrease in bone density, due to a decrease in the function of osteoblasts that make bones due to secretion of hormones from fat by insulin deficiency and metabolism, and in type 2 diabetic patients, it is caused by a decrease in insulin secretion function and a decrease in activity with age, and bone density is similar to or increased in general people, but the decline in bone quality plays a major role. The main mechanisms that cause osteoporosis in diabetes are decreased function of osteoblasts due to insulin deficiency, decreased function of osteoblasts involved in bone formation, and dysfunction of osteoclasts involved in bone metabolism. The detailed mechanisms of osteoporosis in type 1 and type 2 diabetes include hyperglycemia, hyperlipidemia, adipokine and endocrine changes, and an increase in the number of osteoclasts accompanied by inflammation, resulting in an increase in bone resorption and decreased bone formation due to dysfunction and decreased number of osteoblasts, decreased new blood vessels in bone, reduced bone formation due to abnormal differentiation of mesenchymal cells and a decrease in bone quality due to an increase in advanced glycation end-products. The relationship between diabetes mellitus and osteoporosis is a subject of controversy because bone concentration is reduced by more than 50% in type 1 diabetes and increased bone concentration in type 2 diabetes mellitus. In particular, the increase in the risk of fracture in type 2 diabetes is mainly due to the deterioration in the quality of bone tissue, suggesting the possibility that adiponectin inhibits osteoclasts and stimulates osteoblasts to increase bone formation, resulting in a bone protective effect in osteoporosis. Other studies have reported that adiponectin stimulates RANKL (receptor activator of nuclear factor-KB ligand) and inhibits RANKL's decoy receptor OPG (osteoprotegerin) in osteoclasts to induce osteoclastogenesis and inhibit bone formation.

As a treatment method for diabetic osteoporosis, diabetic diet, calcium supplementation and exercise are used, and bisphosphonate preparations, fluoride preparations, or growth hormones are used for drug treatment for men and elderly women, and calcitonin preparations are used for pain caused by spinal compression fractures. A recent study showed that the use of peroxisome proliferator-activated receptor (PPAR)-γ ligand, an oral antidiabetic drug, has a negative effect on bone metabolism and increases the risk of fracture, especially in elderly women. In addition, it has been reported that incretin glucagon-like peptide (GLP)-1, glucose-dependent insulinotropic polypeptide (GIP) preparations, and dipeptidyl peptidase (DPP)-4 inhibitors reduce the relative risk of fracture. The problem is that incretin therapy is known to increase the risk of osteoporotic fractures even if bone mass is maintained.

Postmenopausal osteoporosis is associated with a decrease in bone density after menopause, and increases mainly after the age of 50, resulting in spinal and radial fractures, and it is a very important health problem that as senile osteoporosis progresses, fractures of the femur, proximal humerus, ankle, and pelvis occur, and can cause serious disability to patients and even lead to death. The social cost, including indirect costs, caused by postmenopausal osteoporosis (including senile osteoporosis) in the elderly aged 65 or older in Korea is up to KRW 1.165 trillion over the past 5 years. Control of lifestyle-related factors such as calcium and vitamin D intake, exercise, fall prevention, smoking cessation, sobriety, and nutritional management are important treatment methods. Drug therapy includes selective estrogen receptor modulators (Raloxifene, Bazedoxifene, etc.), bisphosphonides (Alendronate, Risedronate, etc.), RANKL monoclonal antibodies (Denosumab, etc.), There is parathyroid hormone (Teriparatide; Teriparatide, etc.), but drugs cause mild digestive disorders to severe electrolyte metabolism abnormalities. Existing treatments suppress bone loss but do not restore lost bone mass and cause fatal complications when taken for a long time, so natural products such as traditional foods or herbal medicines are trying to find a solution.

[Prior Patent Literature]

Korean Patent Publication No. 2017-0066476

### DISCLOSURE

### Technical Problem

The present invention has been made to solve the above problems, and an object of the present invention is to provide a new pharmaceutical composition for preventing or treating diabetic or postmenopausal osteoporosis.

### Technical Solution

To achieve the above object, the present invention provides a pharmaceutical composition for preventing or treating diabetic or postmenopausal osteoporosis, comprising adiporon as an active ingredient.

In addition, the present invention provides a health functional food for preventing or improving diabetic or postmenopausal osteoporosis containing adiporon as an active ingredient.

The present invention also provides a method for treating diabetic or postmenopausal osteoporosis comprising administering a therapeutically effective amount of adiporon to a patient in need thereof.

The present invention will be described below.

The present inventors was completed by confirming that. adiporon, an oral receptor ligand that selectively acts on adiponectin-receptor 1/2, activates adiponectin-receptor 1-AMPK, a cell metabolism regulator, and activates adiponectin-receptor 2-PPARα, regardless of the improvement of hyperglycemia and dyslipidemia, and through the activation, it improves lipotoxicity, inflammatory response and cell death in bone through the reduction of adipose tissue accumulation, inflammatory response and oxidative stress in bone, and through growth plate promotion effect and chondrocyte proliferation effect in growth plate, and through above effects, diabetic and postmenopausal osteoporosis can be prevented and treated.

Adiporon is a selective, oral synthetic substance having the following structure. It acts on adiponectin-receptor 1 and 2, activating AMP-activated protein kinase (AMPK) and PPARα signaling system, respectively, to be involved in insulin resistance, dyslipidemia, and glucose metabolism.

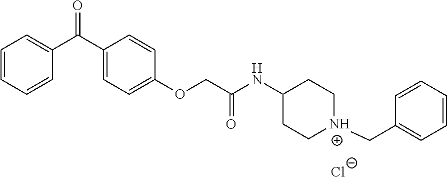 [SMILES: O=C(COc1ccc(C(=O)c2ccccc2)cc1)NC1CC[NH+](Cc2ccccc2)CC1 ; [Cl-]]

AMPK is an enzyme related to cellular energy homeostasis and is a key metabolic regulator regulating various intracellular systems including glucose uptake. AMPK activated under metabolic stress blocks the process of consuming ATP and NADPH, such as protein and fatty acid synthesis, and activates the process of producing them, such as fatty acid degradation, to maintain energy and redox homeostasis, ultimately regulates survival and death of cell. Decreased sensitivity of AMPK activity to cellular stress impairs metabolic control, increases oxidative stress, and reduces autophagy. As such, AMPK plays an important role in the process of metabolic regulation through uncoupling protein (UCP-1).

As shown in FIG. 1, the adiponectin may increase the expression of the adiponectin-receptor 1/2 to continuously activate the adiponectin-receptor 1/2-AMPK-PPARα signaling pathway but is not limited thereto. The adiporon reduces adipose tissue in the bone and reduces inflammation and oxidative stress through the activation of adiponectin-receptor 1-AMPK and adiponectin-receptor 2-PPARα, which are cell metabolism regulators, regardless of the improvement of hyperglycemia and dyslipidemia, thereby reducing lipotoxicity, inflammatory response, and cell death indicators, but are not limited thereto.

The adiporon increases indicators for bone volume, bone surface density, percent bone volume, trabecular thickness, trabecular number and bone mineral density in ilium and vertebrae and decreases the trabecular interval, thereby improving the indicators for osteoporosis, but is not limited thereto.

The adiporon may increase and improve an index of growth plate thickness in ilium, but is not limited thereto.

The adiporon may increase and improve the expression of adiponectin-receptor 1/2, AMPK, PPARα and PGC-1α in ilium and spine, but is not limited thereto. Specifically, the adiporon may activate the adiponectin-receptor 1-AMPK-Nrf2 signaling system and the adiponectin-receptor 2/PPARα-PGC-1a signaling system, but is not limited thereto.

The adiporon may reduce adipocytes and RANKL in ilium and spine but is not limited thereto. In addition, the adiporon can reduce inflammatory response through reduction of nuclear factor erythroid 2 related factor 2 (Nrf2), superoxide dismutase (SOD) 1/2, NAD(P)H: quinone oxidoreductase (NQO) 1 and heme oxygenase (H0)-1 and oxidative stress of NADPH oxidase(NOX)4, and reduction of nuclear factor (NF)-kB and tumor necrosis factor (TNF)-α, and reduce Apoptosis by terminal deoxynucleotidyl transferase dUTP nick end labeling (TUNEL) and can reduce apoptosis by reduction of terminal deoxynucleotidyl transferase dUTP nick end labeling (TUNEL) but is not limited thereto.

In addition, the adiporon may increase serum acid phosphatase (ACP)5(ACP)5: tartrate-resistant acid phosphatase) and osteocalcin concentrations and decrease urine deoxypyridinoline concentration in diabetic and postmenopausal osteoporosis animal models, but is not limited thereto.

The pharmaceutical composition according to the present invention may be prepared in the form of incorporating the active ingredient into a pharmaceutically acceptable carrier. Here, the pharmaceutically acceptable carrier includes carriers, excipients and diluents commonly used in the pharmaceutical field. Pharmaceutically acceptable carriers usable in the pharmaceutical composition of the present invention include, but are not limited to, lactose, dextrose, sucrose, sorbitol, mannitol, xylitol, erythritol, maltitol, starch, gum acacia, alginate, gelatin, calcium phosphate, calcium silicate, cellulose, methyl cellulose, polyvinyl pyrrolidone, water, methyl hydroxybenzoate, propyl hydroxybenzoate, talc, magnesium stearate and mineral oil.

The pharmaceutical composition of the present invention may be formulated and used in the form of oral formulations such as powders, granules, tablets, capsules, suspensions, emulsions, syrups, aerosols, external preparations, suppositories or sterile injection solutions according to conventional methods, respectively.

When formulated, it may be prepared using diluents or excipients such as commonly used fillers, extenders, binders, wetting agents, disintegrants, and surfactants. Solid preparations for oral administration include tablets, pills, powders, granules, capsules, etc., and such a solid preparation may be prepared by mixing active ingredients with at least one or more excipients, for example, starch, calcium carbonate, sucrose, lactose, gelatin, and the like. In addition to simple excipients, lubricants such as magnesium stearate and talc may also be used. Liquid preparations for oral administration include suspensions, solutions for oral administration, emulsions, syrups, etc. In addition to commonly used diluents such as water and liquid paraffin, various excipients such as wetting agents, sweeteners, aromatics, and preservatives may be included. Formulations for parenteral administration include sterilized aqueous solutions, non-aqueous solvents, suspensions, emulsions, freeze-dried formulations and suppositories. Propylene glycol, polyethylene glycol, vegetable oils such as olive oil, and injectable esters such as ethyl oleate may be used as non-aqueous solvents and suspending agents. As a base for suppositories, witepsol, tween 61, cacao oil, laurin oil, glycerogelatin, and the like may be used.

The pharmaceutical composition according to the present invention can be administered to a subject by various routes. All modes of administration are contemplated, e.g., oral, intravenous, intramuscular, subcutaneous, intraperitoneal injection.

The dosage of the pharmaceutical composition according to the present invention is selected in consideration of the age, weight, gender, and physical condition of the subject. It is obvious that the concentration of the active ingredient included in the pharmaceutical composition can be variously selected according to the subject, and is preferably included in the pharmaceutical composition at a concentration of 0.01 to 5,000 μg/ml. If the concentration is less than 0.01 μg/ml, pharmacological activity may not appear, and if the concentration exceeds 5,000 μg/ml, toxicity to the human body may be exhibited.

The pharmaceutical composition may be formulated into various oral or parenteral dosage forms.

Formulations for oral administration include, for example, tablets, pills, hard and soft capsules, solutions, suspensions, emulsifiers, syrups, granules, etc. In addition to active ingredients, these formulations contain diluents (e.g., lactose, dextrose, sucrose, mannitol, sorbitol, cellulose and/or glycine), lubricants (e.g., silica, talc, stearic acid and its magnesium or calcium salts and/or or polyethylene glycol) may be further included. In addition, the tablet may comprise a binder such as magnesium aluminum silicate, starch paste, gelatin, tragacanth, methylcellulose, sodium carboxymethylcellulose and/or polyvinylpyrrolidone, and it may optionally contain disintegrants or effervescent mixtures such as starch, agar, alginic acid or its sodium salt and/or absorbents, colorants, flavors, and sweeteners. The formulation may be prepared by conventional mixing, granulating, or coating methods.

In addition, a typical formulation for parenteral administration is an injection formulation, and water, Ringer's solution, isotonic physiological saline or suspension may be used as a solvent for the injection formulation. Sterile fixed oils of the above injectable preparations may be used as a solvent or suspension medium, and any bland fixed oil may be used for this purpose, including mono- and di-glycerides. In addition, the formulation for injection may use a fatty acid such as oleic acid.

In addition, the present invention provides a health functional food for preventing or improving diabetic or postmenopausal osteoporosis containing adiporon as an active ingredient.

In addition to comprising the active ingredient, the food composition of the present invention may comprise various flavoring agents or natural carbohydrates as additional ingredients like conventional food compositions.

Examples of the above-mentioned natural carbohydrates include monosaccharides such as glucose, fructose, and the like; disaccharides such as maltose, sucrose and the like; and polysaccharides such as conventional sugars such as dextrin and cyclodextrins, and sugar alcohols such as xylitol, sorbitol and erythritol. As the flavoring agents described above, natural flavoring agents (thaumatin), stevia extracts (e.g., rebaudioside A, glycyrrhizin, etc.) and synthetic flavoring agents (saccharin, aspartame, etc.) can advantageously be used. The food composition of the present invention can be formulated in the same way as the pharmaceutical composition and used as a functional food or added to various foods. Foods to which the composition of the present invention can be added include, for example, beverages, meat, chocolate, foods, confectionery, pizza, ramen, other noodles, gum, candy, ice cream, alcoholic beverages, vitamin complexes and health supplements, etc.

In addition, the food composition, in addition to extracts as active ingredients, may comprise various nutrients, vitamins, minerals (electrolytes), flavors such as synthetic flavors and natural flavors, colorants and enhancers (cheese, chocolate, etc.), pectic acid and its salts, alginic acid and its salts, organic acids, protective colloidal thickeners, pH adjusting agents, stabilizers, preservatives, glycerin, alcohol, carbonating agents used in carbonated beverages, and the like. In addition, the food composition of the present invention may comprise fruit flesh for preparing natural fruit juice, fruit juice beverages, and vegetable beverages.

The functional food composition of the present invention can be prepared and processed in the form of tablets, capsules, powders, granules, liquids, pills, and the like. In the present invention, ‘health functional food composition’ refers to a food manufactured and processed using raw materials or ingredients having functional properties useful for the human body according to Health Functional Food Act No. 6727, and it refers to intake for the purpose of obtaining useful effects for health purposes such as regulating nutrients or physiological functions for the structure and function of the human body. The health functional food of the present invention may comprise ordinary food additives, and the suitability as a food additive is determined according to standards and items according to the general rules of the Food Additive Code and general test methods approved by the Food and Drug Administration, unless otherwise specified. Examples of the items listed in the ‘Food Additive Code’ include, for example, chemical compounds such as ketones, glycine, calcium citrate, nicotinic acid, and cinnamic acid; natural additives such as persimmon pigment, licorice extract, crystalline cellulose, kaoliang pigment, and guar gum; and mixed preparations such as sodium L-glutamate preparations, noodle-added alkali preparations, preservative preparations, and tar color preparations. For example, a health functional food in the form of a tablet is obtained by granulating a mixture obtained by mixing the active ingredient of the present invention with excipients, binders, disintegrants, and other additives in a conventional manner, and then compression molding by adding a lubricant or the like. The mixture can be directly compression molded. In addition, the health functional food in the form of a tablet may contain a flavoring agent and the like as needed. Among health functional foods in the form of capsules, hard capsules can be prepared by filling a mixture in which the active ingredient of the present invention is mixed with additives such as excipients in a normal hard capsule and soft capsules can be prepared by filling a mixture obtained by mixing the active ingredient of the present invention with additives such as excipients into a capsule base such as gelatin. The soft capsule may contain a plasticizer such as glycerin or sorbitol, a colorant, a preservative, and the like, if necessary. The health functional food in the form of a pill can be prepared by molding a mixture of the active ingredient of the present invention mixed with an excipient, a binder, a disintegrant, etc. by a conventionally known method, and can be coated with sucrose or other coating agent if necessary, Alternatively, the surface may be coated with a material such as starch or talc. Health functional food in the form of granules can be prepared in granular form by a conventionally known method of mixing the active ingredient of the present invention with excipients, binders, disintegrants, etc., and, if necessary, flavoring agents, etc.

In addition, the present invention provides a method for diabetic or postmenopausal osteoporosis comprising administering a pharmaceutical composition comprising adiporon as an active ingredient to a subject in need of treatment for diabetic or postmenopausal osteoporosis.

The treatment method of the present invention includes administering to a subject a therapeutically effective amount of a pharmaceutical composition containing the same as an active ingredient. A specific therapeutically effective amount for a specific individual depends on the type and extent of the response to be achieved, the specific composition including whether other agents are used as the case may be, the age, weight, general health condition, sex and diet of the individual, the time of administration, It is preferable to apply differently according to various factors including the route of administration and secretion rate of the composition, treatment period, drugs used together with or concurrently used with the specific composition, and similar factors well known in the medical field. Therefore, the effective amount of the composition suitable for the purpose of the present invention is preferably determined in consideration of the above.

The subject is applicable to any mammal, and the mammal includes not only humans and primates, but also livestock such as cattle, pigs, sheep, horses, dogs, and cats, preferably humans, and particularly adults but is not limited thereto.

### Advantageous Effects

As can be seen from the present invention, the present invention can help in screening for colorectal cancer and advanced adenoma by substituting the expression patterns of genetic markers expressed in blood into an artificial intelligence algorithm using a relatively easy-to-extract blood sample.

The composition of the present invention controls bone-related factors by activating the adiponectin-receptor 1-AMPK-Nrf2 signaling system and the adiponectin-receptor 2/PPARα-PGC-1a signaling system in bone tissue, improves lipotoxicity, and promotes its growth plate and promotes chondrocyte proliferation in the growth plate, and through the above effects, it can be usefully used as a therapeutic agent for diabetic and postmenopausal osteoporosis.

### DESCRIPTION OF DRAWINGS

FIG. 1 shows the mechanism of action of adiporon,

FIG. 2 shows an animal experiment schedule according to an embodiment of the present invention,

FIG. 3 illustrates an animal test schedule according to an embodiment of the present invention.

FIGS. 4 to 14 are tissue changes and micro-CT changes in the ilium in the non-diabetic normal control group db/m mice and type 2 diabetic db/db mouse experimental animals in the group not administered with adiporon and in the case of treatment with adiporon. **P<0.01 (comparison of adiporon-treated and untreated groups in non-diabetic and diabetic groups),

FIGS. 15 to 20 are changes in adiporone receptor 1/2 in the ilium of the non-diabetic control group db/m mice and type 2 diabetic db/db mouse experimental animals in the group not administered with adiporon and in the case of treatment with adiporon. **P<0.01 (comparison of adiporon-treated and untreated groups in non-diabetic and diabetic groups),

FIGS. 21 to 23 show changes in adipocytes and RANKL in the ilium of non-diabetic normal control group db/m mice and type 2 diabetic db/db mouse experimental animals in the group not administered with adiporon and in the case of treatment with adiporon. **P<0.01 (comparison of adiporon-treated and untreated groups in non-diabetic and diabetic groups),

FIGS. 24 to 31 are changes in oxidative stress and antioxidant enzymes in the ilium of non-diabetic normal control group db/m mice and type 2 diabetic db/db mouse experimental animals in the group not administered with adiporon and in the case of treatment with adiporon. **P<0.01 (comparison of adiporon-treated and untreated groups in non-diabetic and diabetic groups),

FIGS. 32 to 34 show the inflammatory response (TNF-α) and changes in apoptosis (TUNEL positive) in the ilium of the non-diabetic normal control group db/m mice and type 2 diabetic db/db mouse experimental animals in the group not administered with adiporon and in the case of treatment with adiporon. **P<0.01 (comparison of adiporon-treated and untreated groups in non-diabetic and diabetic groups),

FIGS. 35 to 42 are lumbar L5 tissue changes and micro-CT changes in a group not administered with adiporon and a group treated with adiporon in experimental animals of non-diabetic normal control group db/m mice and type 2 diabetic db/db mice. **P<0.01 (comparison of adiporon-treated and untreated groups in non-diabetic and diabetic groups),

FIGS. 43 to 49 show the adiporon receptor 1/2 in the L5 lumbar spine in a group not administered with adiporon and a group treated with adiporon in experimental animals of non-diabetic normal control group db/m mice and type 2 diabetic db/db mice. **P<0.01 (comparison of adiporon-treated and untreated groups in non-diabetic and diabetic groups), FIGS. 50 to 52 are changes in adipocytes and RANKL in lumbar spine L5 in a group not administered with adiporon and a group treated with adiporon in experimental animals of non-diabetic normal control group db/m mice and type 2 diabetic db/db mice. **P<0.01 (comparison of adiporon-treated and untreated groups in non-diabetic and diabetic groups),

FIGS. 53 to 61 are changes of oxidative stress and antioxidant enzymes in the lumbar spine L5 in a group not administered with adiporon and a group treated with adiporon in experimental animals of non-diabetic normal control group db/m mice and type 2 diabetic db/db mice. **P<0.01 (comparison of adiporon-treated and untreated groups in non-diabetic and diabetic groups),

FIGS. 62 to 64 show the inflammatory response (TNF-α) and changes in apoptosis (TUNEL positive) in the ilium in a group not administered with adiporon and a group treated with adiporon in experimental animals of non-diabetic normal control group db/m mice and type 2 diabetic db/db mice. **P<0.01 (comparison of adiporon-treated and untreated groups in non-diabetic and diabetic groups),

FIGS. 65 to 66 shows changes of ACP5/TRAP and osterocalcin concentrations in serum deoxypyridinoline (DPP) in urine in a group not administered with adiporon and a group treated with adiporon in experimental animals of non-diabetic normal control group db/m mice and type 2 diabetic db/db mice. **P<0.01 (comparison of adiporon-treated and untreated groups in non-diabetic and diabetic groups),

FIGS. 67 to 79 show changes of tissue and micro-CT in the iliac crests in normal control groups and ovariectomized mouse experimental animals in which adiporon was not administered and when adiporon was treated at 2.5 mg/kg body weight (2.5 AdiR) and 25 mg/kg body weight (25 AdiR). In addition, changes in the thickness of the growth plate of the ilium of ovariectomized mice were investigated (trichrome staining, ×400). In addition, the growth plate thickness of the ilium of ovariectomized mice and changes in chondrocytes in the growth plate were investigated (trichrome staining, Safranin O staining ×400) *P<0.05, **P<0.01 (control group and ovariectomy group, adiporon treatment group and non-treatment group comparison),

FIGS. 80 to 87 show changes in adiporone receptor 1/2, AMPK, RANKL, PGC-1α, and OPG in the iliac crests in normal control groups and ovariectomized mouse experimental animals in which adiporon was not administered and 2.5 mg/kg body weight (2.5 AdiR) and 25 mg/kg body weight (25 AdiR) were treated. *P<0.05, **P<0.01 (comparison between adiporon-treated and non-treated groups in the control and ovariectomized groups), FIGS. 88 to 96 show changes of tissue changes in the lumbar vertebrae L5 and micro-CT, in normal control groups and ovariectomized mouse experimental animals in which adiporon was not administered and adiporon was treated at 2.5 mg/kg body weight (2.5 AdiR) and 25 mg/kg body weight (25 AdiR). *P<0.05, **P<0.01 (comparison of adiporon treated and untreated groups in control and ovariectomized groups),

FIGS. 97 to 104 show changes in adiporon receptor 1/2, AMPK, RANKL, PGC-1α, and OPG in the lumbar vertebrae L5 of normal control and ovariectomized mouse experimental animals treated with 2.5 mg/kg body weight (2.5 AdiR) and 25 mg/kg body weight (25 AdiR) of adiporon and a group not administered with adiporon. *P<0.05, **P<0.01 (comparison of adiporon treated and untreated groups in control and ovariectomized groups), and

FIGS. 105 to 107 show changes of ACP5/TRAP and osterocalcin concentrations in serum and deoxypyridinoline (DPP) in urine in a group in normal control and ovariectomized mouse experimental animals treated with 2.5 mg/kg body weight (2.5 AdiR) and 25 mg/kg body weight (25 AdiR) of adiporon and a group not administered with adiporon. *P<0.05, **P<0.01 (comparison of adiporon treated and untreated groups in control and ovariectomized groups).

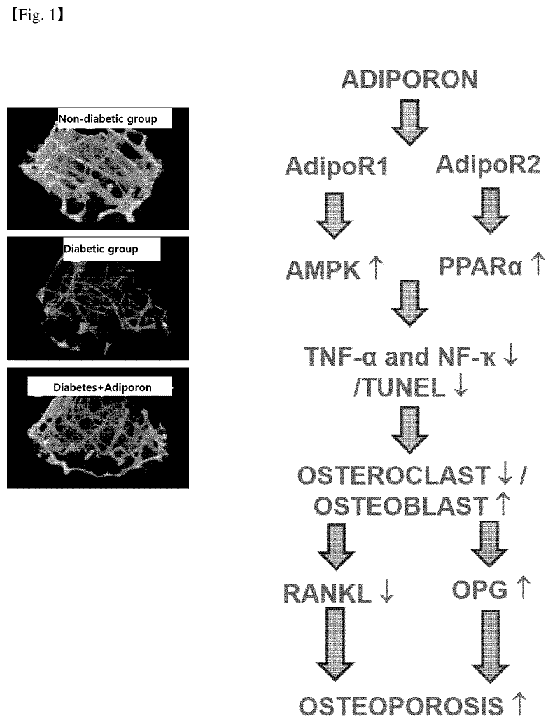

### MODE FOR INVENTION

Hereinafter, the present invention will be described in more detail by the following examples. However, the following examples are described with the intention of illustrating the present invention, and the scope of the present invention is not to be construed as being limited by the following examples.

### Example

### Example 1. Experimental method

### Evaluation of the Effectiveness for the Treatment and Prevention of Diabetic Osteoporosis of Oral Adiporon

### <1> Animal Testing 1

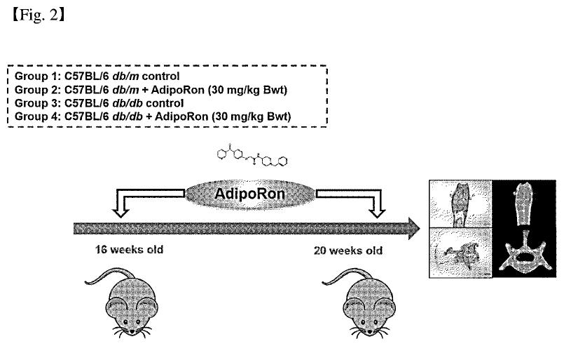

As shown in FIG. 2, animal experiments were conducted. A leptin receptor-defective type 2 diabetic animal model (db/db mice) and a normal control group (db/m mice) were used, and the experiment was performed by dividing into 4 groups as follows: non-diabetic control group (dm cont, n=8), non-diabetic adiporon treatment group (dm+AdipoR, n=8), diabetic control group (db cont, n=8) and diabetic adiporon treatment group (db+AdipoR, n=8).

The control group was fed a normal diet, and the adiporon treatment group was fed a diet comprising adiporon (30 mg/kg/day) for 4 weeks from 16 weeks of age. During the experiment, body weight was measured every week, fasting blood glucose was measured using an Accu-Chek meter (Roche Diagnostics, St. Louis, MO) every 2 weeks after blood was collected from the tail vein, and glycated hemoglobin (HbA1c) was measured every 4 weeks using a Pfizer 1200 automatic analyzer (Bayer healthcare LLC, IN) after blood was collected from the tail vein. The temperature and humidity of the breeding room were maintained at 20˜25° C. and 50˜60%, respectively, and the lights were turned on and off at 12-hour intervals.

### Animal Testing 2

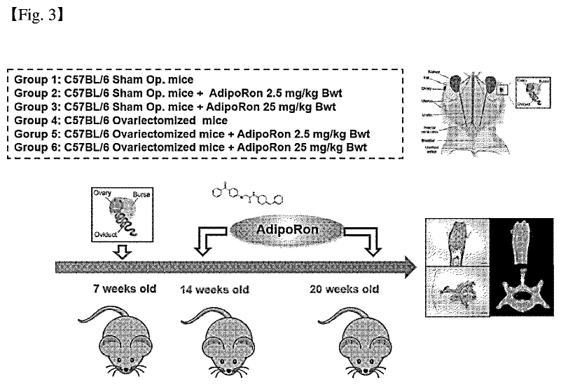

As shown in FIG. 3, animal experiments were conducted. 7 weeks after ovariectomization or sham surgery of 7-week-old C57BL/6 mice, normal feed group (adiporon non-administered group; control group) administration group, 2.5 mg/kg day or 25 mg/kg day, respectively Feed comprising adiporon was consumed for 6 weeks from 14 weeks of age. Body weight was measured every week during the experiment period, and the experiment was conducted after 6 weeks of administration.

### <2> Biochemical Test in Serum and 24-Hour Urine

In both experiments, blood collected from each mouse was allowed to stand at room temperature for 30 minutes, and then centrifuged at 3000 rpm for 15 minutes to obtain serum. Blood levels of tartrate-resistant acid phosphatase (TRAP/ACP5) and osterocalcin concentration were measured by ELISA. In addition, 8-hydroxy-deoxyguanosine, a DNA damage marker caused by oxidative stress, and isoprostane, a marker of oxidative stress caused by lipid peroxidation caused by free fatty acids, and deoxypyridinoline (DPP), which indicates the function of osteoclasts, were measured in 24-hour urine concentrations by ELISA method.

### <3> Micro-CT Scan

The imaging was performed with micro-CT (Skyscan 1172, Belgium), the tube voltage was 60 kV, the tube current was 167 uA, 0.5 mm aluminum filtration was used, and the pixel size was 5.9 μm. For the shooting angle, a 2-dimensional image was reconstructed with Nrecon Reconstruction (Skyscan, Belgium). For 3D image analysis, CTAn (Skyscan, Belgium) was used, images were obtained by rotating 360°, and exposure time was 440 ms. A total of 7 types of indicators were used in this invention. BMD (bone mineral density, bone density) was measured based on phantoms of 0.25 g/cm³ and 0.75 g/cm³. BV/TV (Bone volume/Total volume, percent bone volume, %) is the ratio of voxels representing the solid region out of the total voxels present in the binarized and surfaced volume of interest, and it means the ratio occupied by bone trabeculae within the volume of interest, and i.s (Interception surfaces, mm2) means the generation of new bone. i.s (Interception surfaces, mm²) means the creation of new bone. BS/BV (Bone surface/Bone volume, bone specific surface, mm⁻¹) is the ratio of the voxel surface area to the number of voxels in the binarized solid region within the volume of interest and it means the ratio of the surface area of the trabecular bone to the volume of the trabecular bone. The lower the value, the higher the bone strength. Tb.Th (trabecular thickness, mm) is obtained by averaging the diameters of these spheres, after placing a sphere comprising the corresponding voxel for each voxel representing the solid region within the volume of interest so that the size of the sphere is the largest that includes only the solid region and it means the average thickness of the trabecular bone. After making it possible, it is obtained by averaging the diameters of these spheres, which means the average thickness of the trabecular bone. In the same way, using Tb.Sp (trabecular separation, mm), the average length between trabecular lines and Tb.N (trabecular number, mm⁻¹) were used to obtain the average number of trabecular lines.

### <4> Histological Examination

After removing tissues of the femur and L5 lumbar vertebrae, some of them were fixed in 10% formalin for immunostaining and decalcified. Thereafter, after neutralization with 0.5M Phosphate buffer (pH 7.4), washing, and embedding in paraffin. Tissue sections were cut to a thickness of 5 μm, and hematoxylin and eosin staining (H&E staining), trichrome staining, and Safranin O staining were performed. In bone tissue, double immunofluorescence staining method was performed using anti-adiponectin receptor 1/2 antibody, anti-perilipin-1 antibody, anti-RANKL antibody, anti-TNF-α antibody, and ApopTag Fluorescein In Situ Apoptosis Detection Kit (Chemicon International, Temecula, CA) and expression was observed using a confocal microscope.

### <5> Western Blot Analysis

Protein was extracted using Pro-Prep Protein Extraction Solution (Intron Biotechnology, Gyeonggi-Do, Korea), and SDS-PAGE (sodium dodecyl sulfate-polyacrylamide gel electrophoresis) was performed. The separated protein was transferred to a nitrocellulose membrane (Amersham Co., Buckinghamshire, England) and treated with Tris buffered saline (in TBS-T: 0.1% Tween-20 in Tris buffer saline, pH 7.5) containing 3% skim milk. After blocking for an hour, the blots were reacted in adiponectin receptor 1/2, total AMPK, phospho-Thr172AMPK, PGC-1α, Nrf-2, SOD1/2, NQO1, HO-1, NOX4, NF-κB and β-actin primary antibody solution, washing, reacting with a secondary antibody against each primary antibody, and then photosensitizing through ECL (Pierce, Rockford IL) to confirm the band. The expression level of each protein was normalized according to β-actin.

### <6> Statistical Analysis

The resulting values were expressed as mean and standard deviation, and the difference between each group was measured using the SPSS 19.0 program (SPSS, Chicago, IL, USA). Comparison of mean values between each group was analyzed using one-way ANOVA and Bonferroni post hoc multiple comparison, and cases with a P value of 0.05 or less were defined as significant.

### Example 2. Experimental Results

### Experiment Result 1

Effects of Adiporon Treatment on Body Weight, Blood Biochemistry, and Oxidative Stress Changes in 24-Hour Urine in Diabetic Osteoporosis Models, db/m and db/db Mice

Body weight, blood glucose, glycated hemoglobin, blood total cholesterol, triglyceride and free fatty acid were significantly increased in db/db mice compared to db/m or db/m+adiporon mice (P<0.001 or P<0.05). In addition, the concentration of adiponectin in blood was significantly decreased in db/db mice compared to db/m or db/m+adiporon mice (P<0.001). These changes were restored by administration of adiporon. In addition, the 24-hour urine concentrations of 8-hydroxy-deoxyguanosine, a DNA damage marker caused by oxidative stress, and isoprostane, a marker of oxidative stress caused by lipid peroxidation by free fatty acids, were significantly increased in db/db mice compared to db/m or db/m+adiporon mice (P<0.001). It was confirmed that these changes improved the index increase with the administration of adiporon (Table 1).

TABLE 1

| | db/m control | db/m + adipoR | db/db control | db/db + adipoR |
|---|---|---|---|---|
| Body wt (g) | 35.5 ± 1.7 | 86.4 ± 1.8 | 56.0 ± 7.3^{c} | 53 ± 4.8^{#} |
| Blood glucose (mg/dl) | 143 ± 11 | 136 ± 8 | 564 ± 65 [symbol] | 549 ± 77 [symbol] |
| HbA1c (%) | 3.9 ± 0.2 | 3.8 ± 0.2 | 10.8 ± 0.4 [symbol] | 11.6 ± 0.5 [symbol] |
| Serum TC (mmol/l) | 2.4 ± 0.3 | 2.5 ± 0.4 | 3.7 ± 0.3 [symbol] | 8.3 ± 0.5 [symbol] |
| Serum TG (mmol/l) | 1.3 ± 0.3 | 1.4 ± 0.2 | 2.3 ± 0.4 [symbol] | 1.7 ± 0.3 [symbol] |
| Serum NEFA (mEq/I) | 0.73 ± 0.22 | 0.67 ± 0.23 | 1.72 ± 0.31 [symbol] | 1.39 ± 0.16 [symbol] |
| Serum adiponectin (ng/ml) | 10126 ± 1090 | 11214 ± 1214 | 5123 ± 456 [symbol] | 5089 ± 782 [symbol] |
| Urinary 8-OHDG (ng/24 h) | 41.4 ± 12.4 | 36.5 ± 14.5 | 219.0 ± 76.9 [symbol] | 74.5 ± 19.6 |
| Urinary isoprostane (ng/24 h) | 4.7 ± 1.3 | 4.3 ± 2.2 | 52.2 ± 16.4 [symbol] | 19.3 ± 9.4* |

[symbol]

indicates data missing or illegible when filed

Effect of Adiporon Treatment on Tissue Changes in the Ilium (Femur) in db/m and db/db Mice

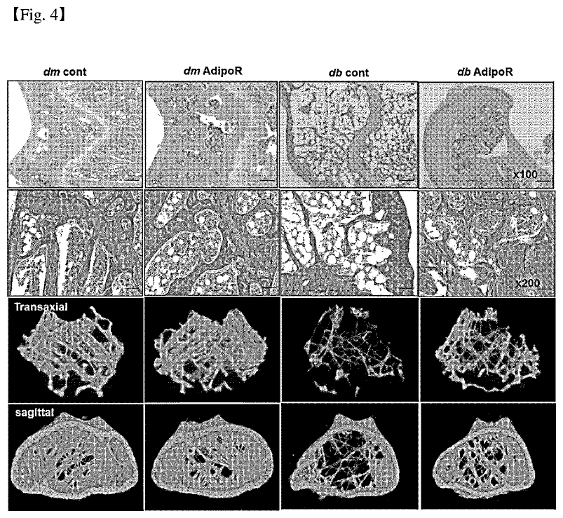

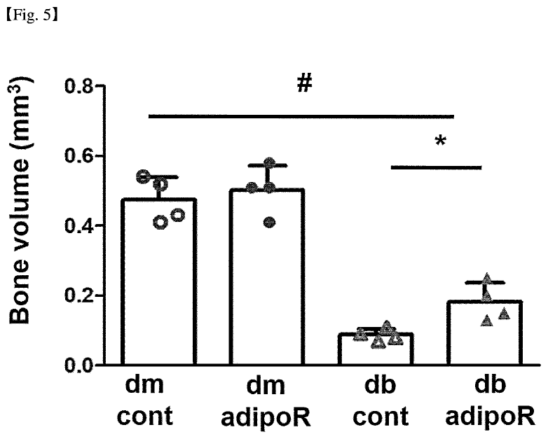

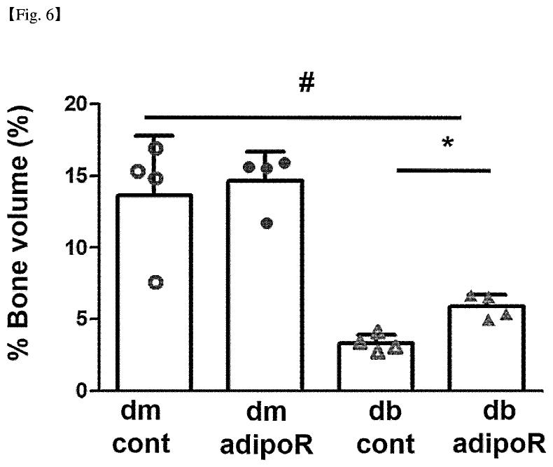

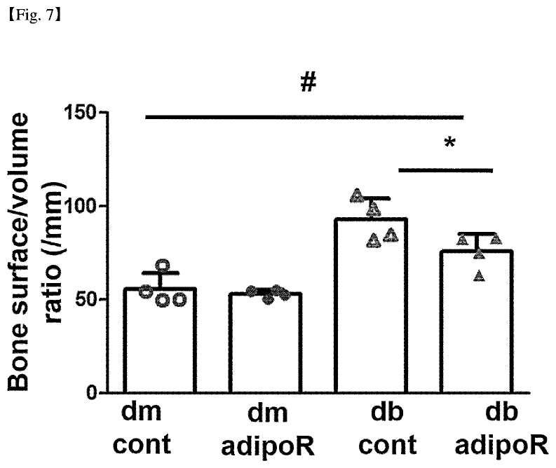

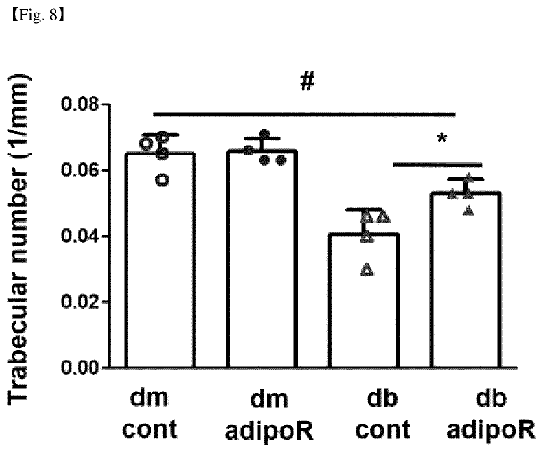

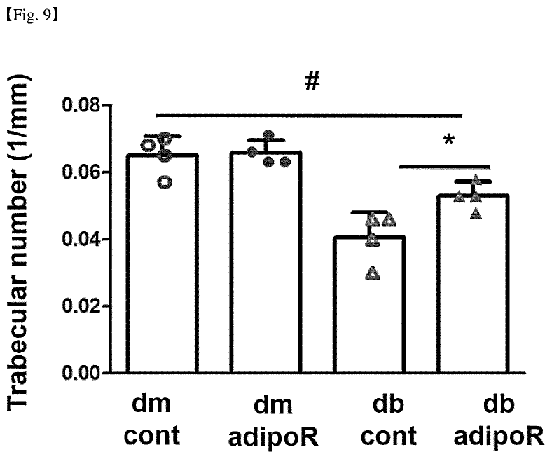

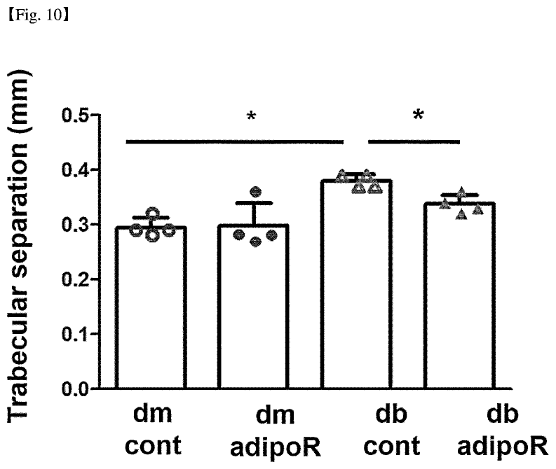

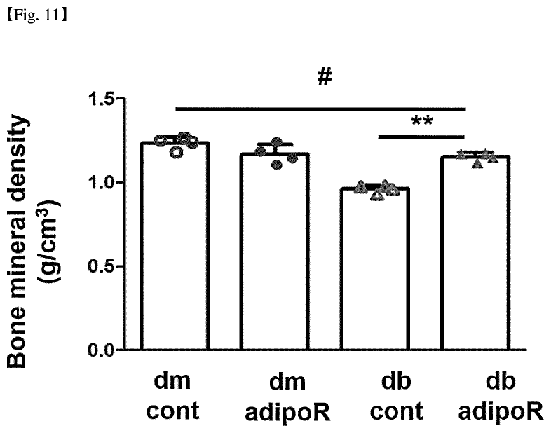

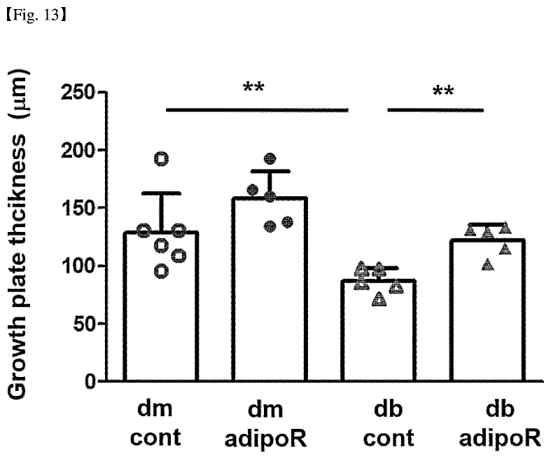

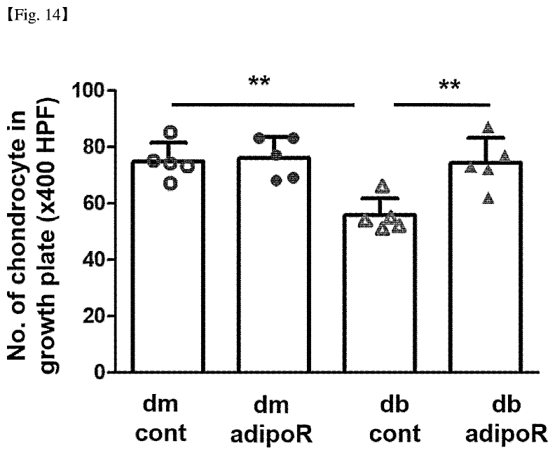

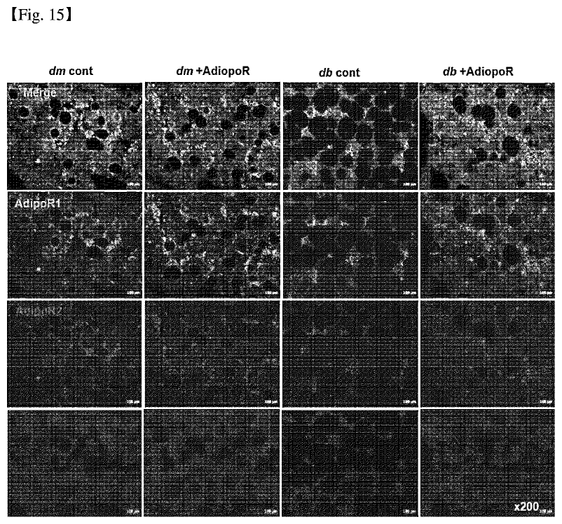

As a result of H&E staining and Safranin O staining in the ilium (femur) tissues of mice, there was a significant increase (P<0.01) in fibrosis and adipose tissue in the tissues of db/db mice compared to db/m or db/m+adiporon mice. Compared to db/m or db/m+adiporon mice, an increase in the thickness of the decreased ilium growth plate of db/db mice was confirmed (P<0.01). The increase in fibrosis and adipose tissue in these tissues and the decrease in the ilium growth plate were recovered by administration of adiporon (FIGS. 4 to 14). In addition, as a result of the micro-CT of ilium tissue, it was confirmed that adiporon administration significantly increased and improved the index for bone volume, bone surface, trabecular thickness, trabecular number, and bone mineral density in tissues of db/db mice (FIGS. 4 to 14).

Effects of Adiporon on the Expression of Adiporon Receptor 1/2 and Intracellular Signaling Pathways in Ilium (Femur) Tissues in db/m and db/db Mice

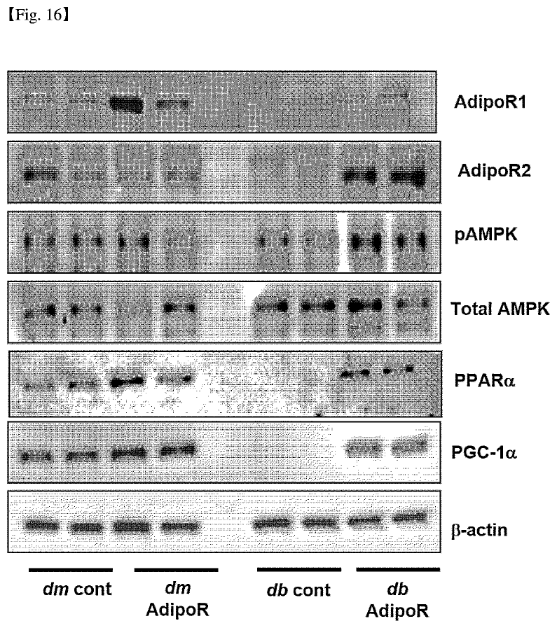

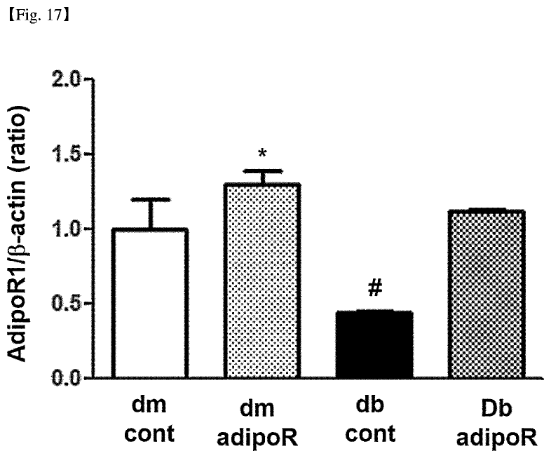

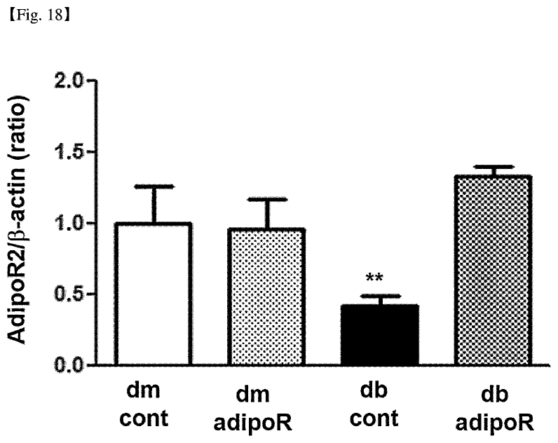

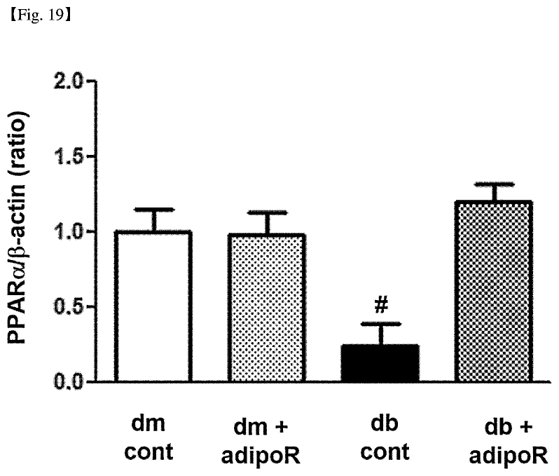

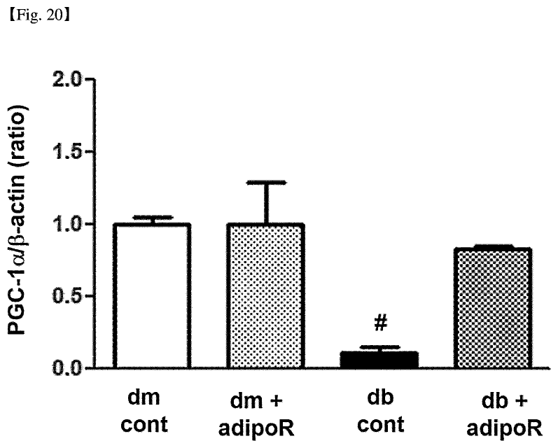

As a result of double immunofluorescence staining of adiponectin-receptor 1/2 in ilium (femur) tissues of mice, the expression in the tissues of db/db mice was significantly reduced compared to db/m or db/m+adiporon mice (P<0.01). This decrease was restored by administration of adiporon. In addition, it was confirmed that the activation of adiponectin-receptor 1-AMPK, a cell metabolism regulator, and the expression of adiponectin-receptor 2-PPARα and PGC1α, which were reduced in db/db mice, were increased by adiporon administration (FIGS. 15 to 20).

Effects of Adiporon on Changes in Adipocytes and RANKL in Ilium (Femur) Tissues in db/m and db/db Mice

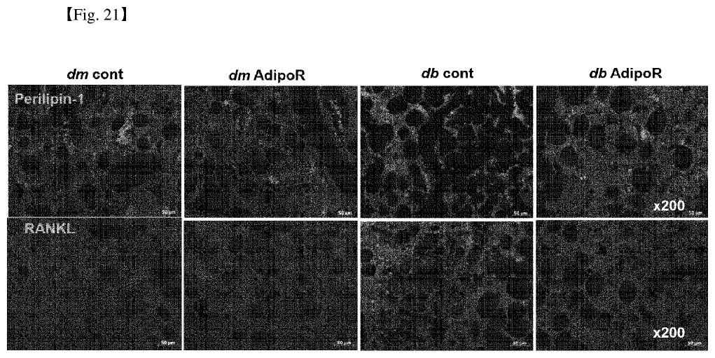

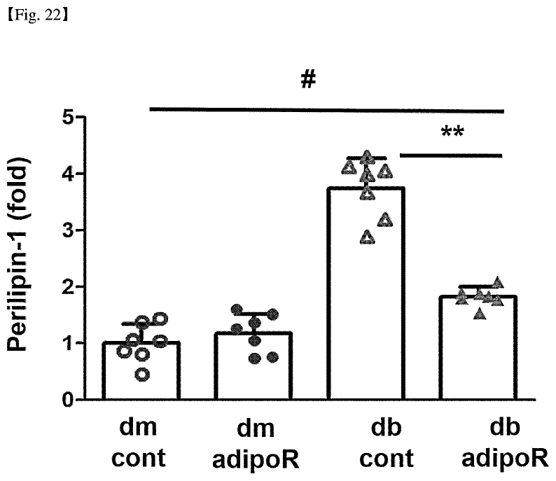

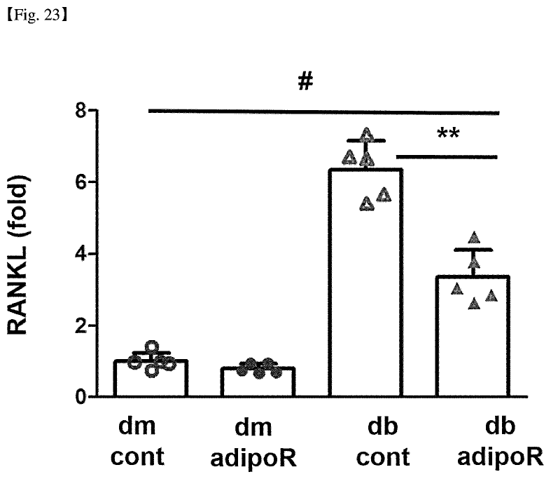

Result of immunofluorescent staining of perilipin-1, an adipocyte marker, in the ilium (femur) tissues of mice showed a significant increase of adipocytes in the tissues of db/db mice compared to db/m or db/m+adiporon mice (P<0.001). This increase in expression was restored by administration of adiporon. In addition, the increased expression of RANKL in db/db mice was restored by administration of adiporon (FIGS. 21 to 23).

Effects of Adiporon on Changes in Oxidative Stress and Antioxidant Enzymes in Ilium (Femur) Tissues in db/m and db/db Mice

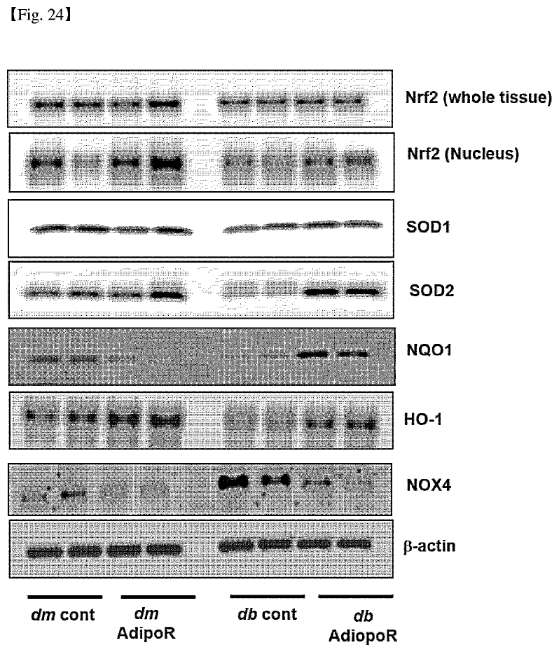

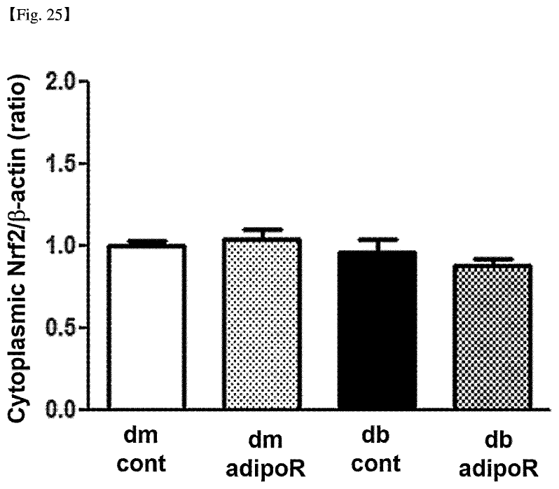

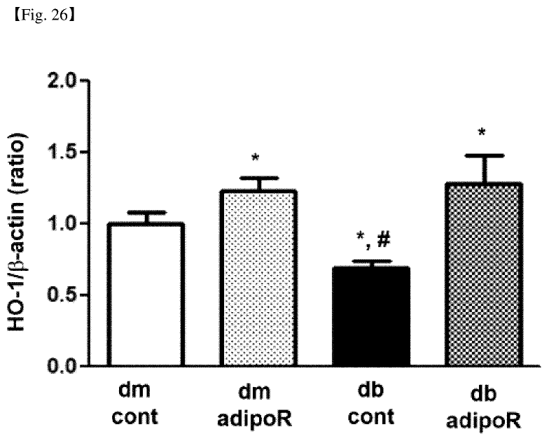

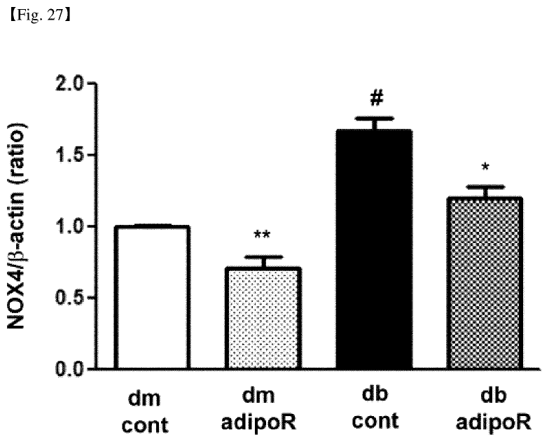

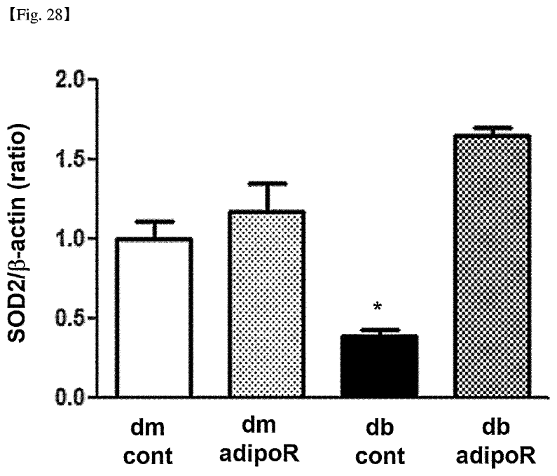

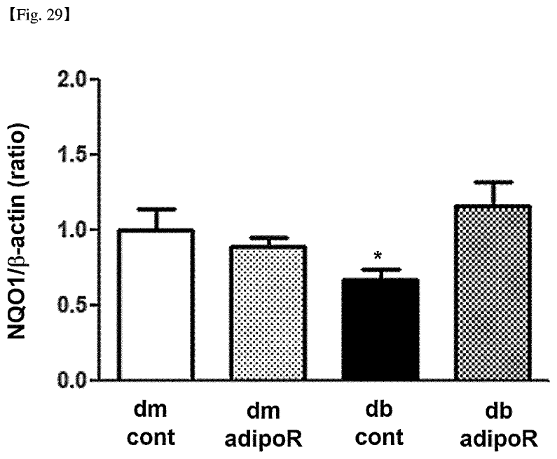

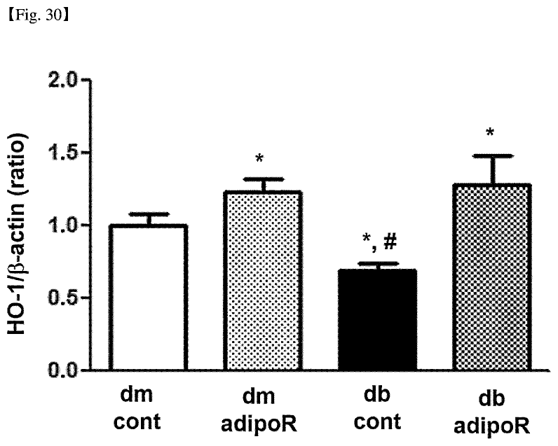

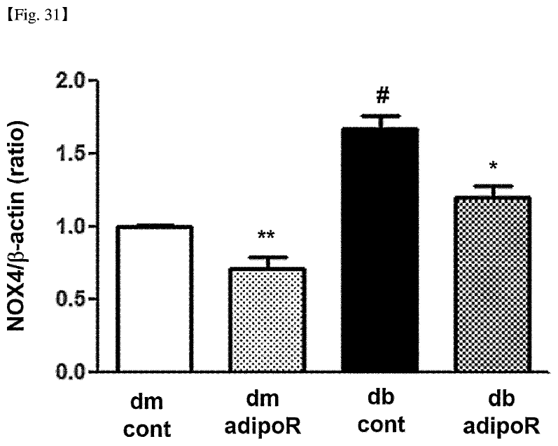

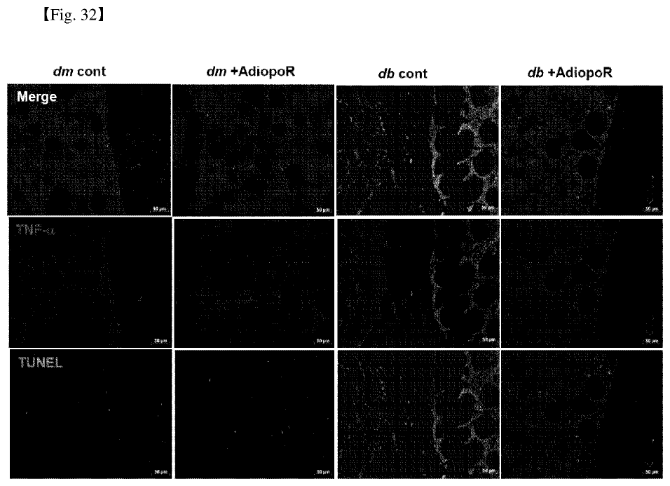

In the present invention, it was confirmed that the expression of oxidative stress markers NOX4 and NFkB in the ilium (femur) increased significantly in db/db mice due to diabetes compared to db/m mice, and it was also confirmed that the expression of the antioxidant enzymes Nrf2, NQO1, HO-1, SOD1 and SOD2 decreased (FIGS. 24 to 31), and it was confirmed that adiporon treatment normalized the expression of oxidative stress markers and antioxidant enzymes in ilium caused by diabetes.

Effects of Adiporon on Ilium (Femur) Inflammatory Response and Apoptosis in db/m and db/db Mice

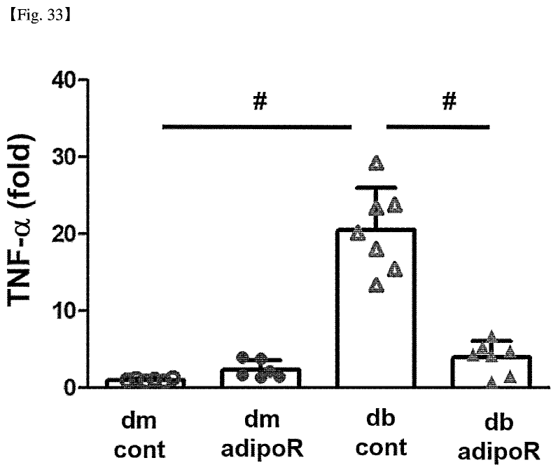

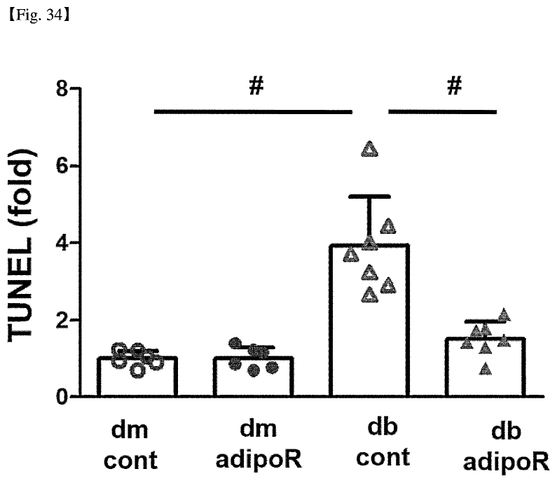

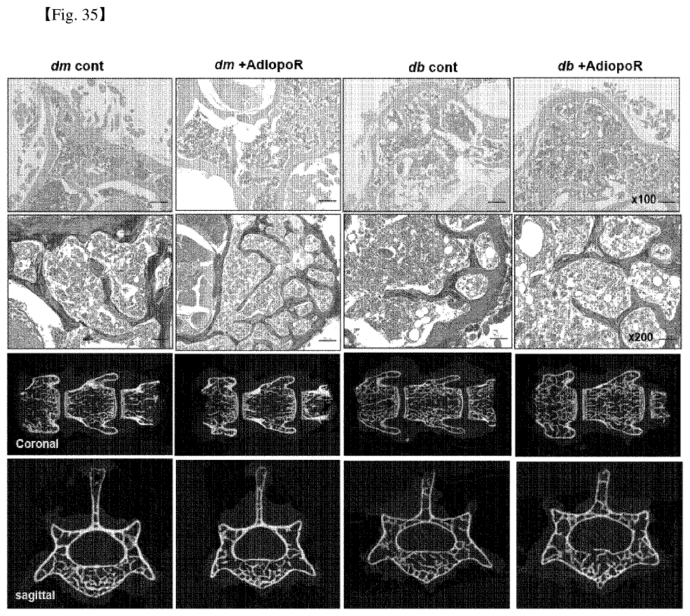

As a result of TNF-α and TUNEL double immunofluorescence staining, which are inflammatory markers, in the ilium (femur) tissues of mice, compared to db/m or db/m+adiporon mice, the expression of TNF-α in the tissue of db/db mice was significantly increased, resulting in increased infiltration of inflammatory cells, and increased TUNEL staining-positive apoptosis (P<0.01). Infiltration of inflammatory cells is known to bring about oxidative stress in ilium, resulting in a vicious cycle of tissue inflammatory response and cell death. This increase in inflammatory response and apoptosis was restored by administration of adiporon (FIGS. 32 to 34).

Effect of Adiporon Treatment on Tissue Changes of Vertebrae (Lumbar Vertebrae 5) in db/m and db/db Mice

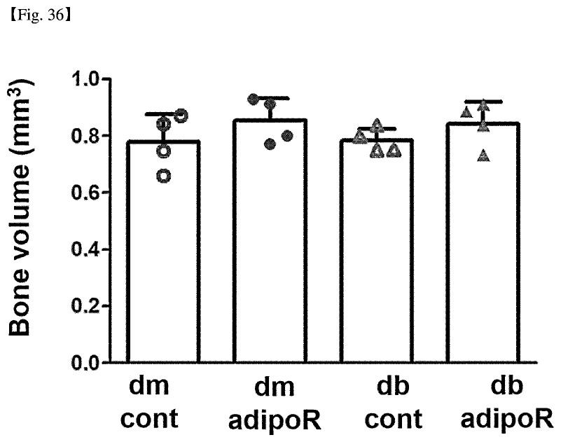

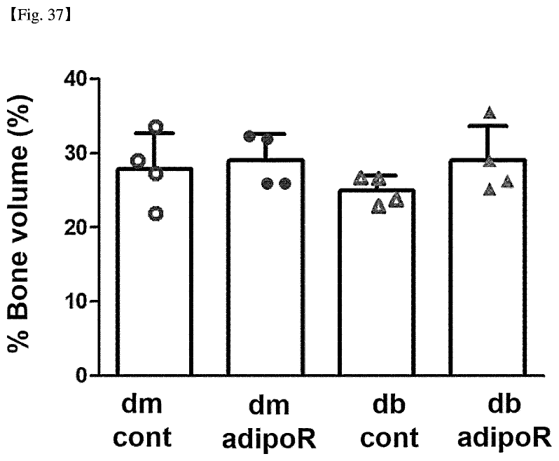

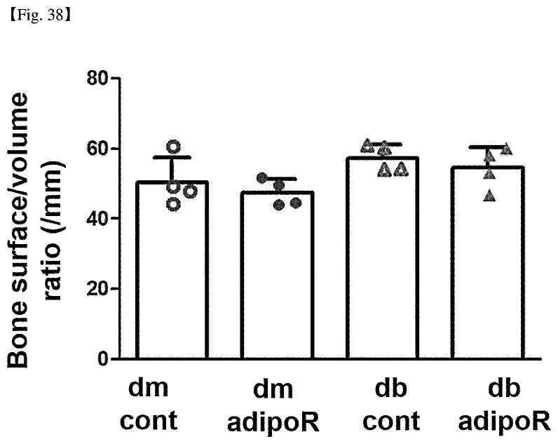

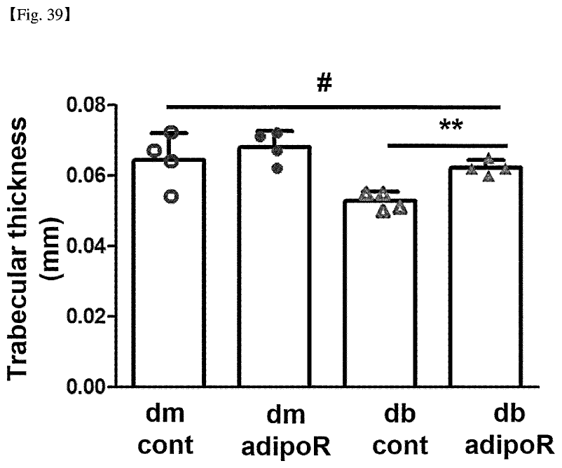

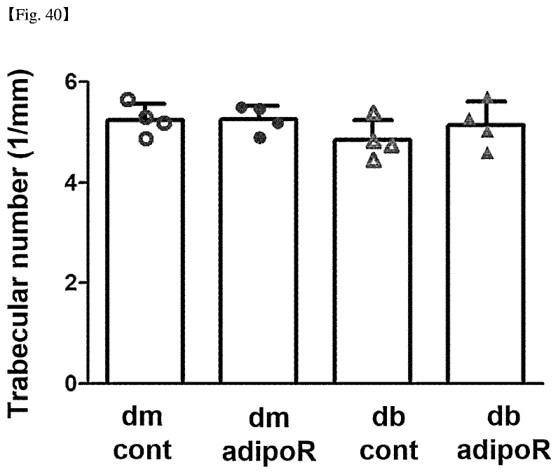

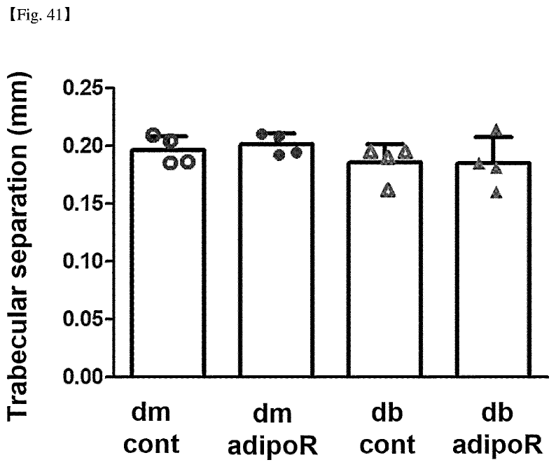

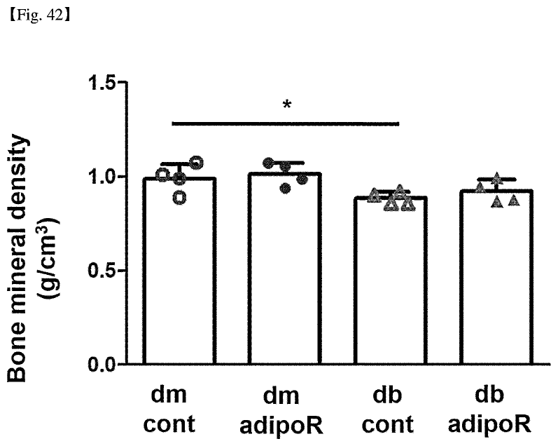

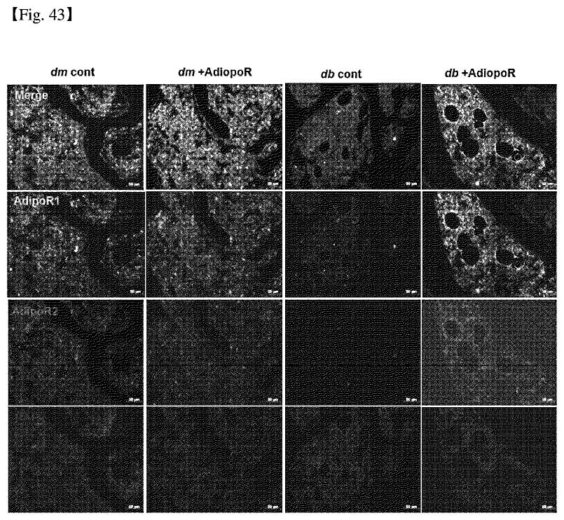

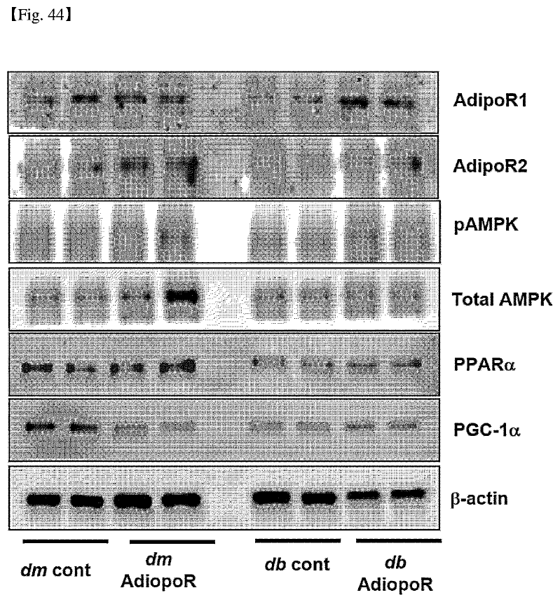

As a result of H&E staining and trichrome staining in the tissues of the vertebral bones (lumbar vertebrae 5) of the mice, fibrosis and adipose tissue in the tissues of db/db mice were increased compared to db/m or db/m+adiporon mice. This increase was reversed by administration of adiporon. In addition, as a result of micro-CT of long bone tissue, it was confirmed that adiporon administration increased and improved the trabecular thickness in the tissue of db/db mice (FIGS. 35 to 42).

Effects of Adiporon on the Expression of Adiporon Receptor 1/2 and Intracellular Signaling Pathways in Vertebral Bone (Lumbar Vertebra 5) Tissues in db/m and db/db Mice

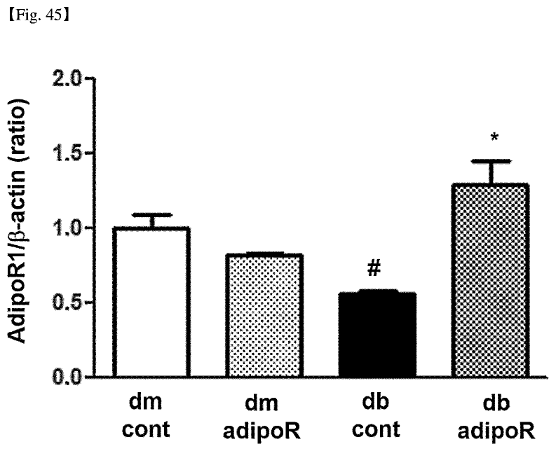

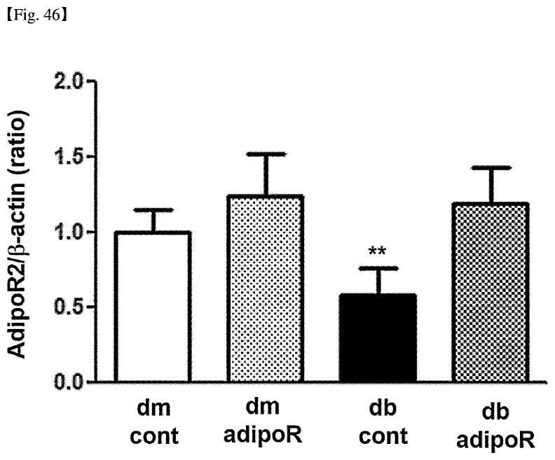

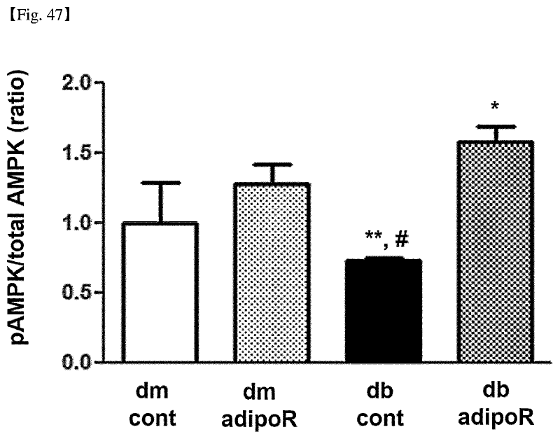

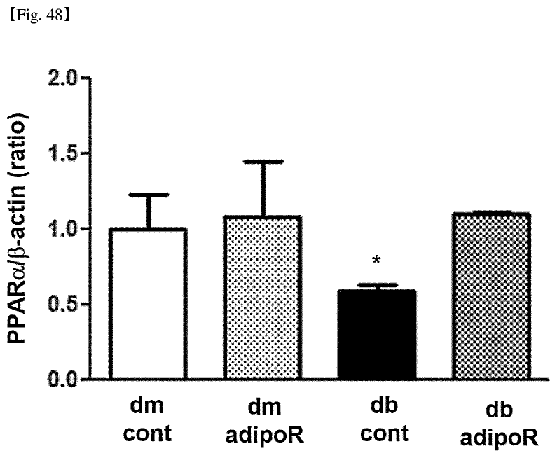

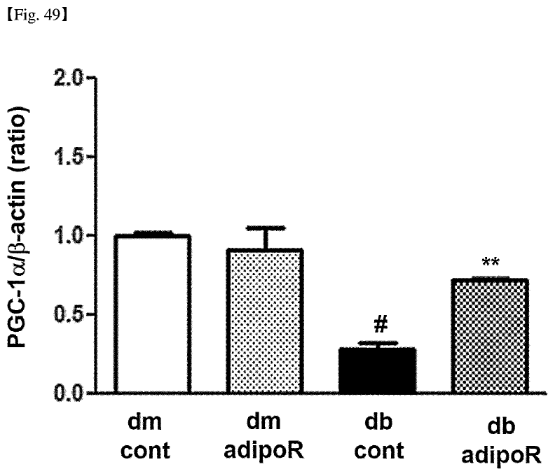

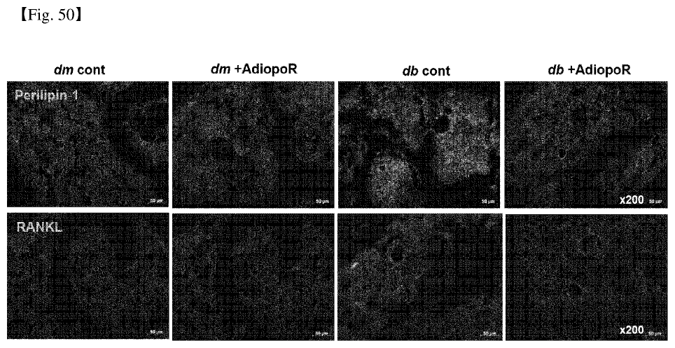

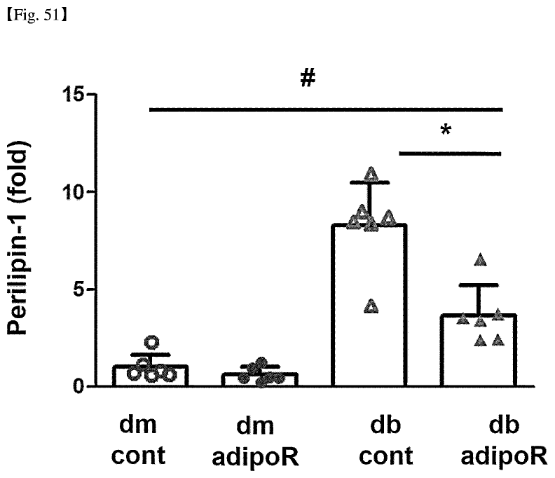

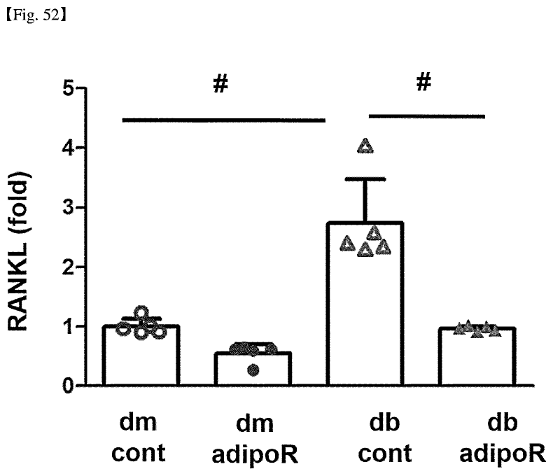

As a result of double immunofluorescence staining of adiponectin-receptor 1/2 in the ilium (lumbar vertebrae 5) tissues of mice, the expression in the tissues of db/db mice was significantly reduced compared to db/m or db/m+adiporon mice (P<0.01). This decrease was reversed by administration of adiporon. In addition, it was confirmed that the activation of adiponectin-receptor 1-AMPK, a cell metabolism regulator, and the expression of adiponectin-receptor 2-PPARα and PGC1α, which were reduced in db/db mice, were increased by adiporon administration (FIGS. 43 to 49).

Effects of Adiporon on Changes in Adipocytes and RANKL in Vertebrae (Lumbar Vertebrae 5) in db/m and db/db Mice

Immunofluorescent staining of perilipin-1, an adipocyte marker, in the tissues of the vertebral bones (lumbar vertebrae 5) of mice showed a significant increase of adipocytes in the tissues of db/db mice compared to db/m or db/m+adiporon mice (P<0.01). This increase in expression was restored by administration of adiporon. In addition, the increased expression of RANKL in db/db mice was restored by administration of adiporon (FIGS. 50 to 52).

Effects of Adiporon on Changes in Oxidative Stress and Antioxidant Enzymes in Vertebrae (Lumbar Vertebrae 5) in db/m and db/db Mice

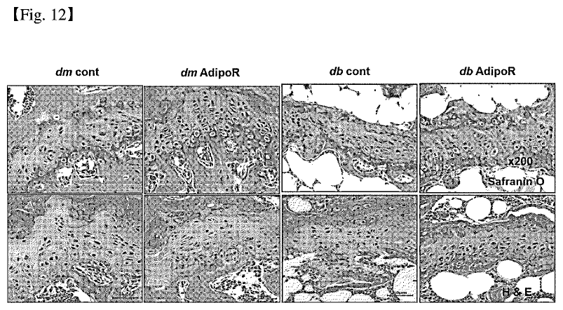

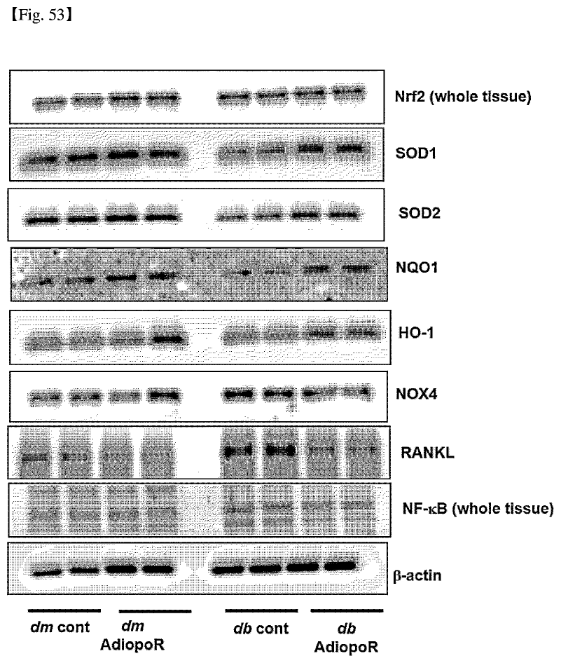

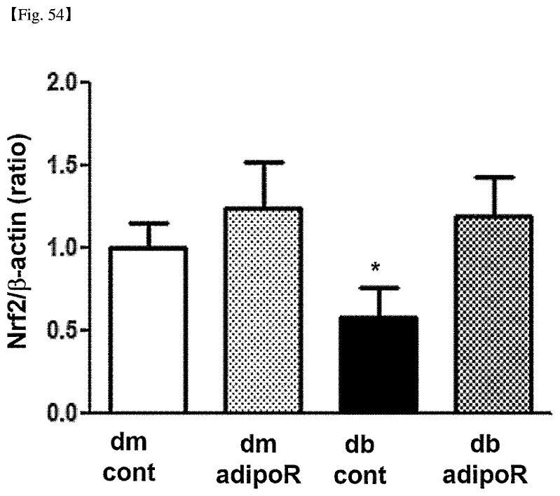

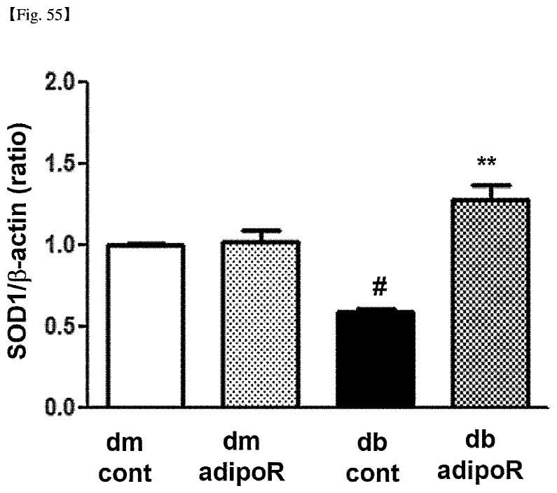

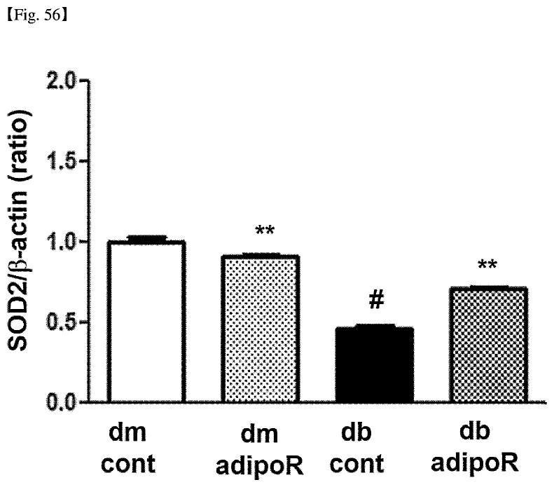

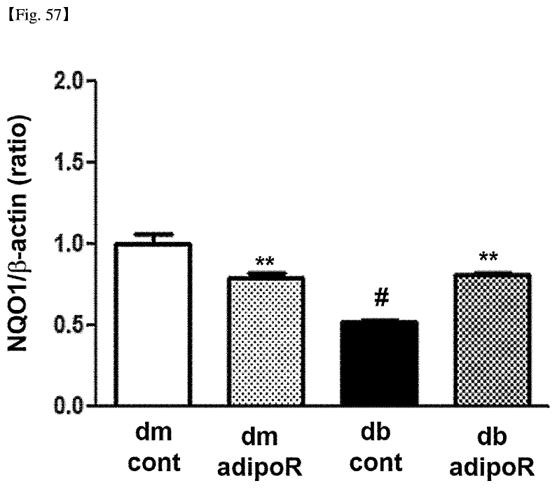

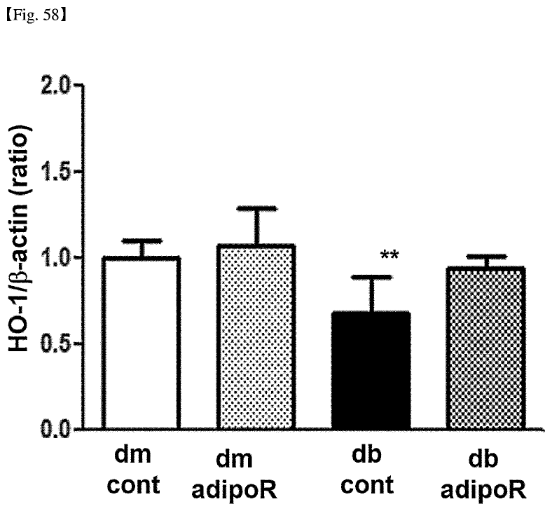

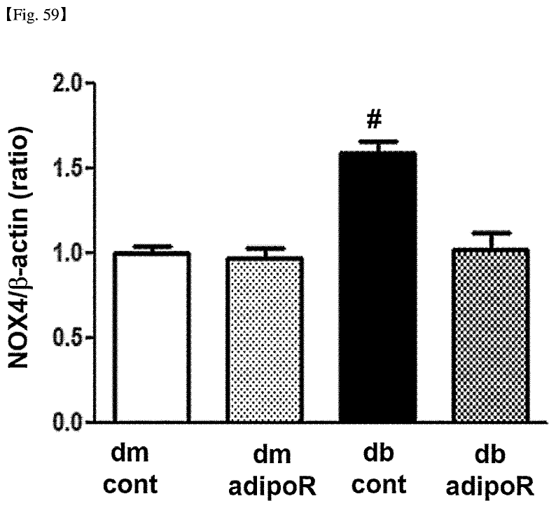

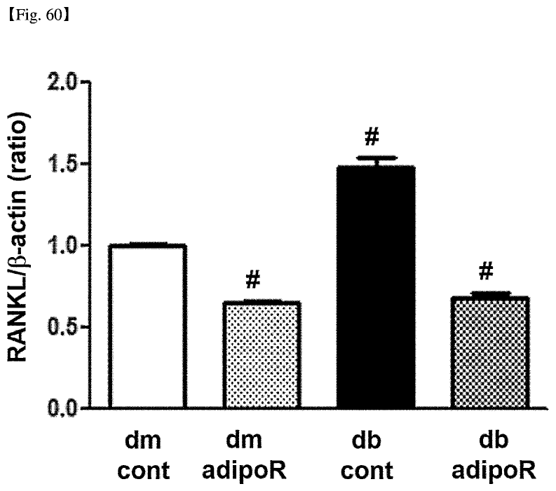

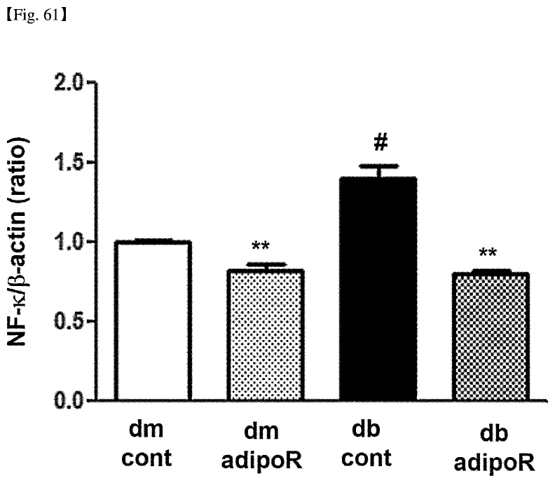

In the present invention, it was confirmed that the expression of oxidative stress markers NOX4 and NFkB in the vertebrae increased in db/db mice compared to db/m mice by diabetes, and it was also confirmed that expression of the antioxidant enzymes Nrf2, NQO1, HO-1, SOD1, and SOD2 decreased (FIG. 12), and it was confirmed that adiporone treatment normalized the expression of oxidative stress markers and antioxidant enzymes in ilium caused by diabetes (FIGS. 53 to 61).

Effects of Adiporon on Inflammatory Response and Apoptosis in Vertebrae (Lumbar Vertebrae 5) in db/m and db/db Mice

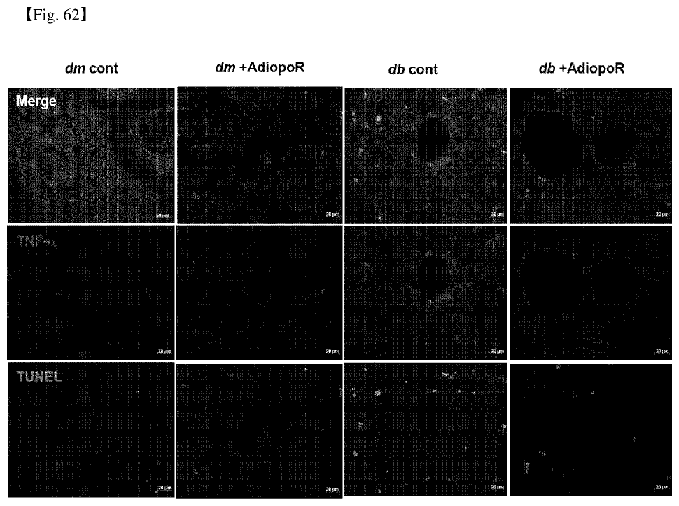

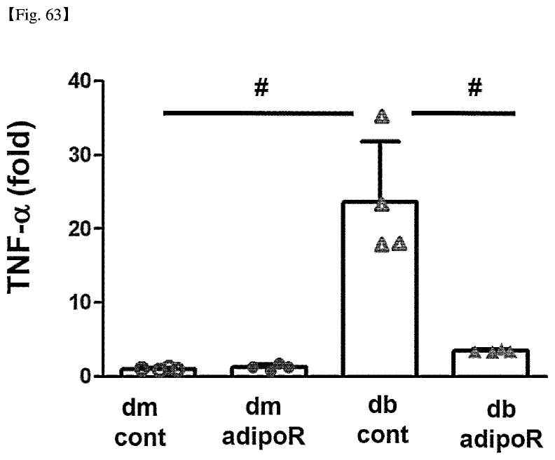

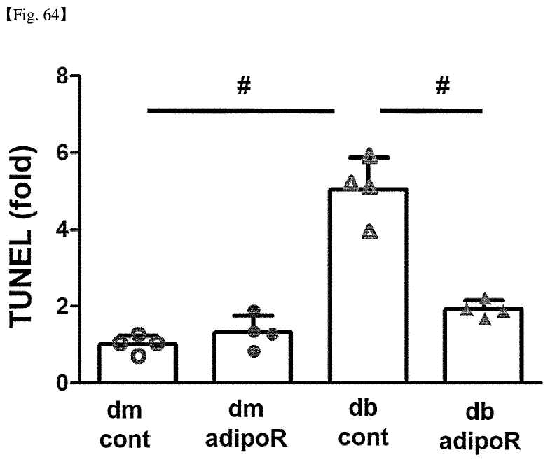

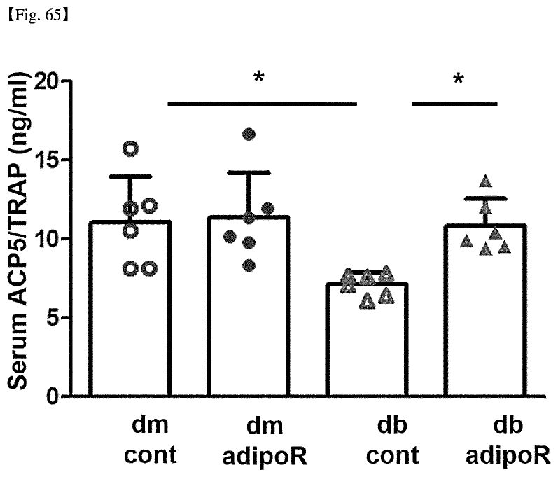

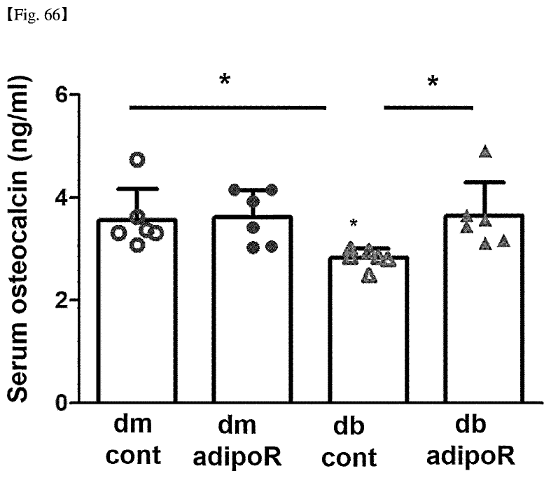

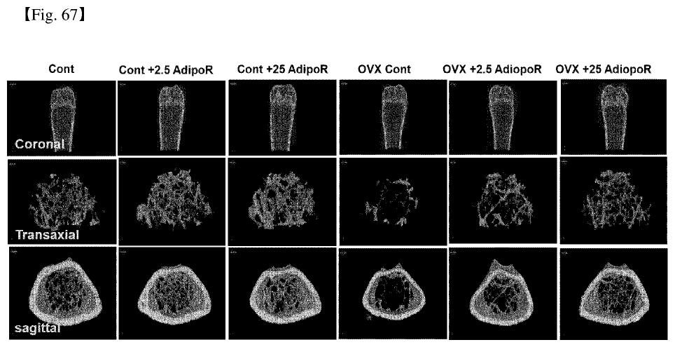

As a result of TNF-α and TUNEL double immunofluorescence staining, which are inflammatory markers, in the vertebral tissue of mice, the expression of TNF-α in the tissues of db/db mice was significantly increased compared to db/m or db/m+adiporon mice, resulting in increasing infiltration of inflammatory cells, and TUNEL staining-positive apoptosis also increased (P<0.001). Infiltration of inflammatory cells is known to bring about oxidative stress in ilium, resulting in a vicious cycle of tissue inflammatory response and cell death. This increase in inflammatory response and apoptosis was restored by administration of adiporon (FIGS. 62 to 64).

Effects of Adiporon on Serum ACP5/TRAP and Osterocalcin Concentrations and Urinary Deoxypyridinoline Changes in db/m and db/db Mice

As a result of confirming serum ACP5/TRAP and osteocalcin concentrations by ELISA method, it was confirmed that the concentrations were significantly decreased in db/db mice compared to db/m or db/m+adiporon mice (P<0.01). This decrease was restored by administration of adiporon (FIGS. 65-66). In addition, the concentration of deoxypyridinoline in urine was significantly increased in db/db mice compared to db/m or db/m+adiporon mice (P<0.001). This increase was reversed by administration of adiporon (P<0.001).

### Experiment Result 2

### Effects of Adiporon Treatment on Tissue Changes in Ilium (Femur) in Ovariectomized Mice

As can be seen from the micro-CT results of the iliac tissue of mice, it was confirmed that the indicators for bone volume, bone surface, trabecular thickness, trabecular number and bone mineral density in the ilium of ovariectomized mice without adiporon administration were increased and improved in 2.5 mg/kg day or 25 mg/kg day adiporon-administered ovariectomy mouse groups, respectively (67 to 79).

### Effects of Adiporon Treatment on Growth Plate Changes in Ilium (Femur) in Ovariectomized Mice

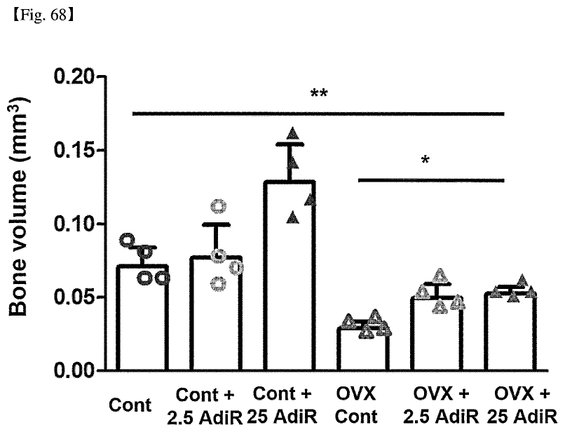

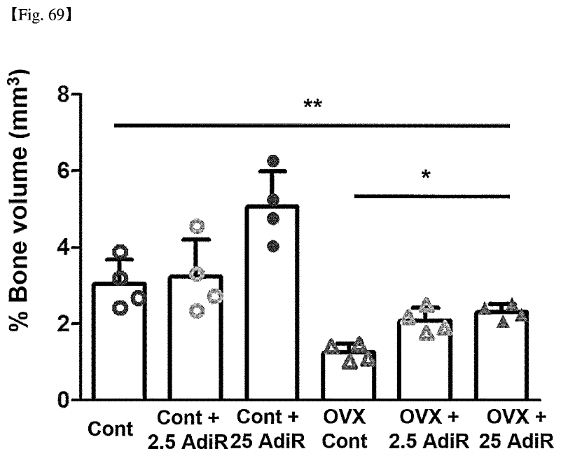

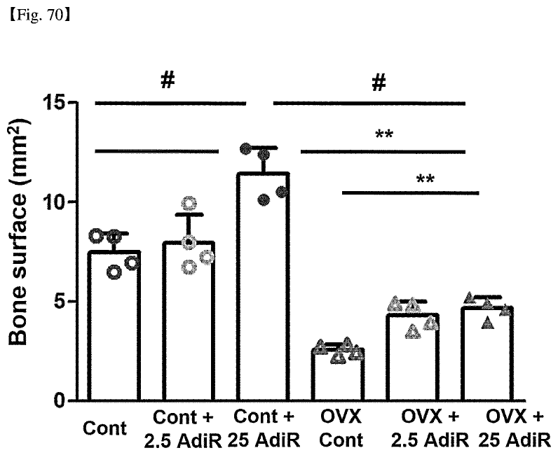

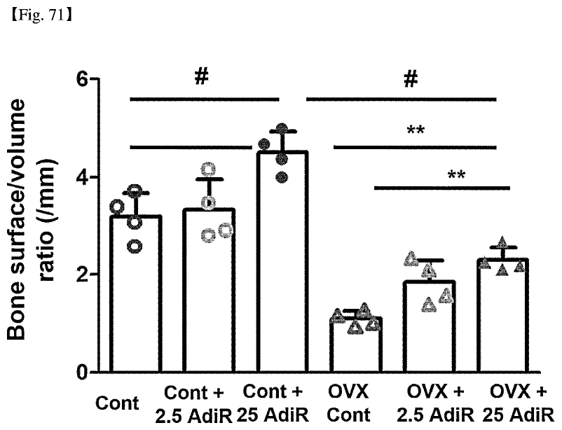

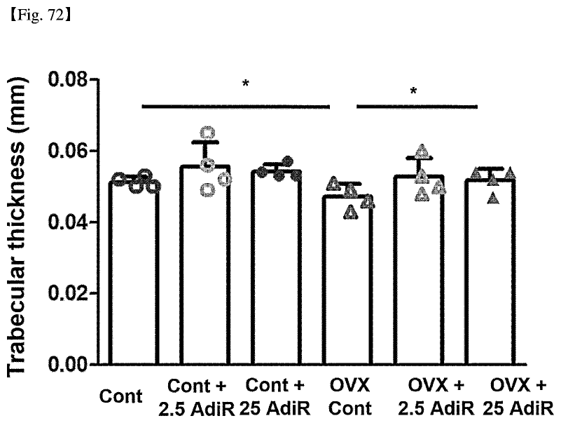

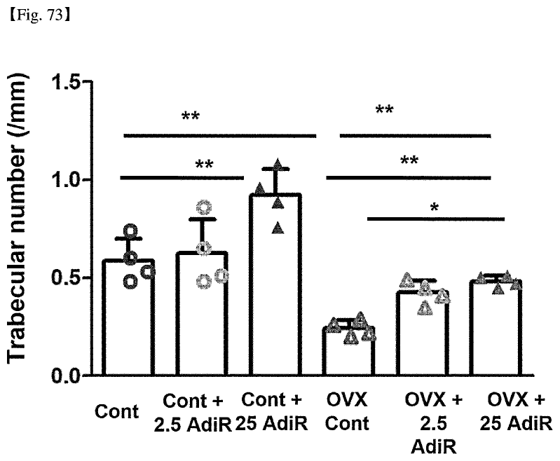

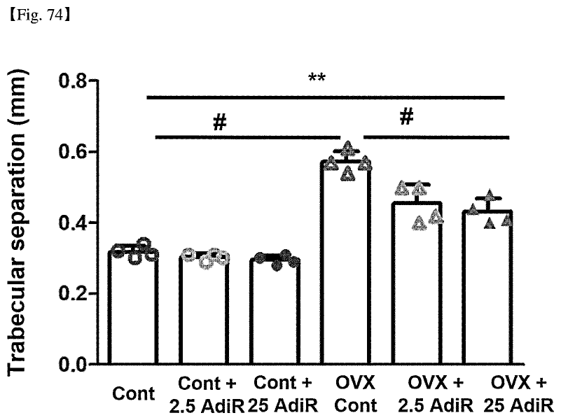

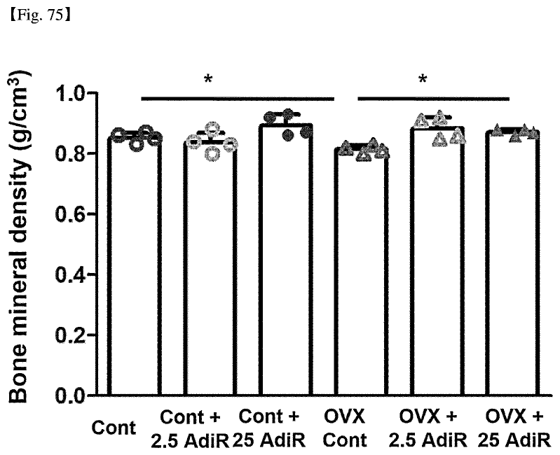

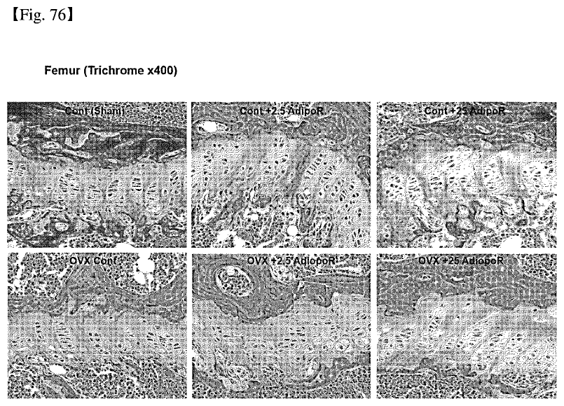

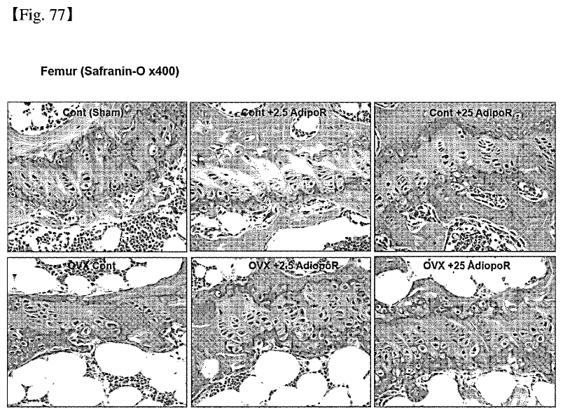

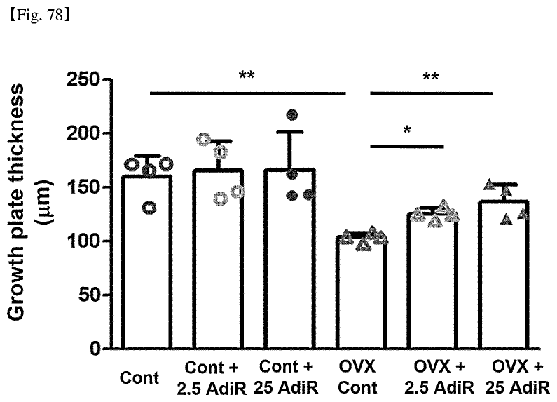

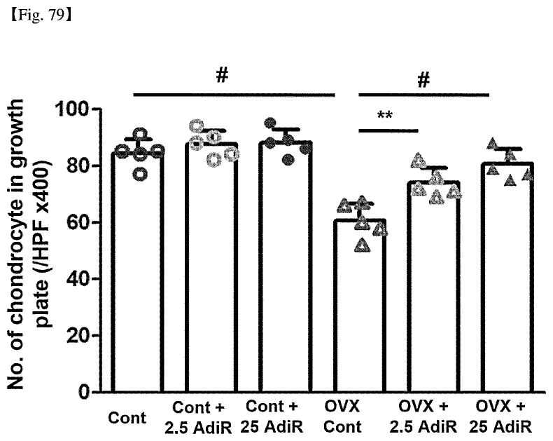

From the staining results (trichrome, Safranin 0) of iliac tissue in mice, in both ovariectomized mouse groups administered with 2.5 mg/kg day or 25 mg/kg day adiporon, respectively, the decreased thickness of the iliac growth plate in the ovariectomized mouse group increased, confirming the protective effect of improving the reduction of the ilium growth plate (FIGS. 67 to 79).

### Effects of Adiporon Treatment on Expression of Adiporon Receptor 1/2 and Intracellular Signal Transduction System in Ilium (Femur) Tissues in Ovariectomized Mice

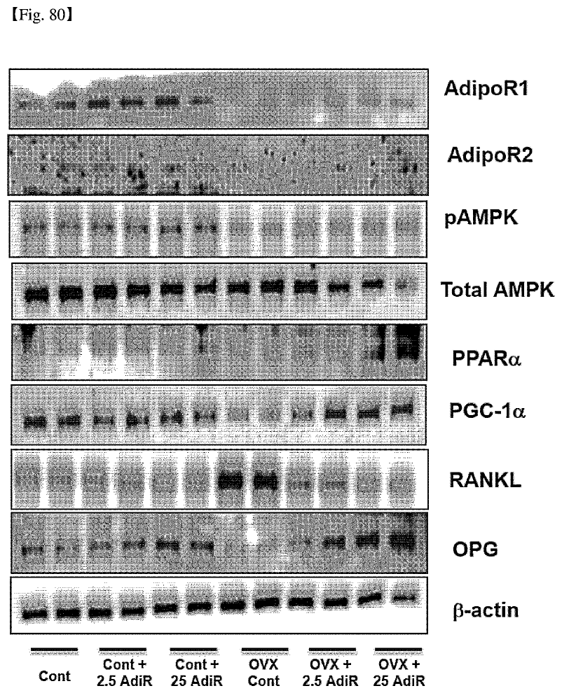

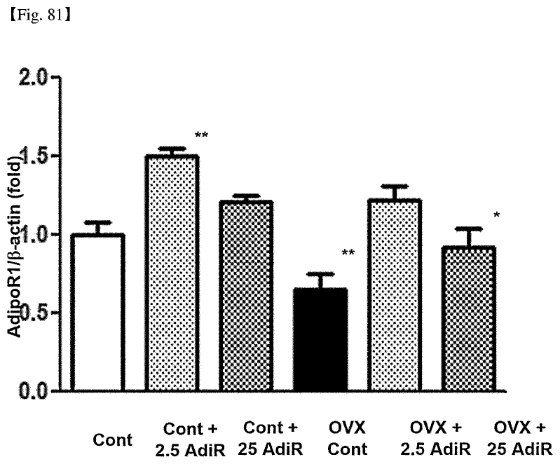

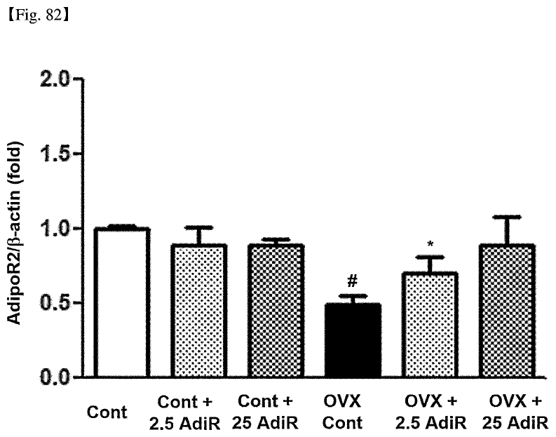

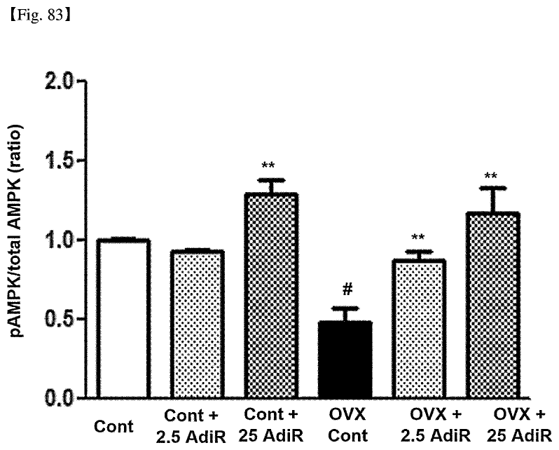

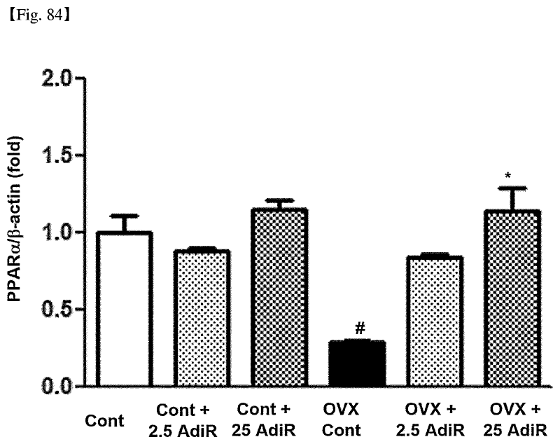

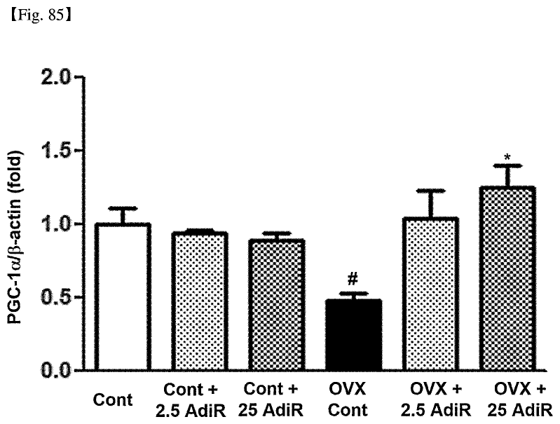

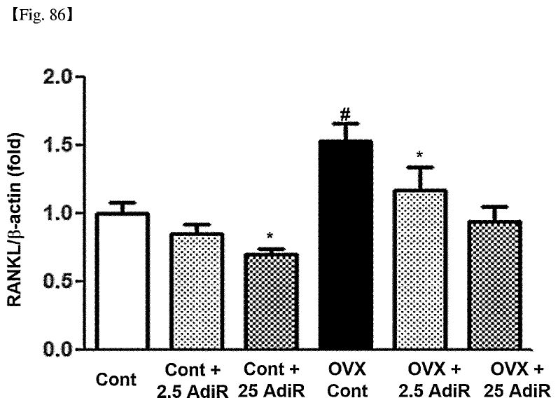

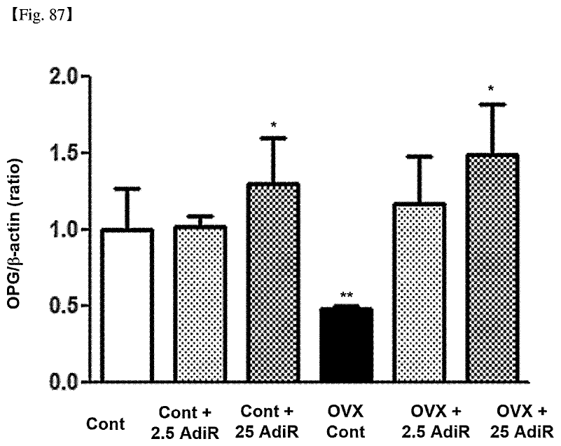

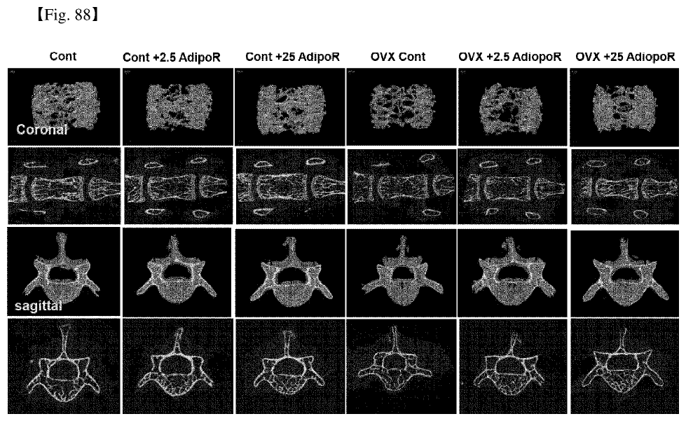

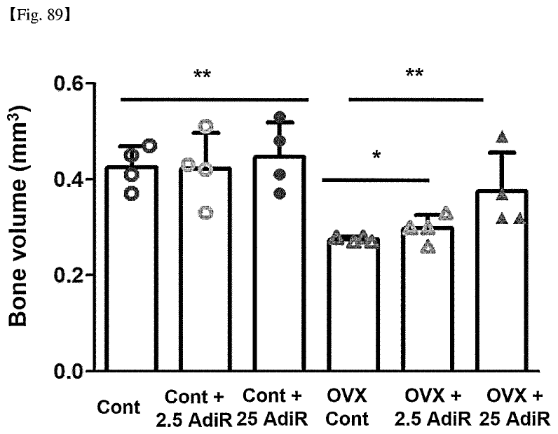

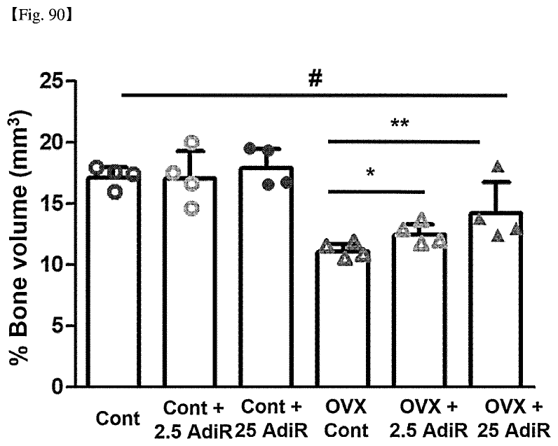

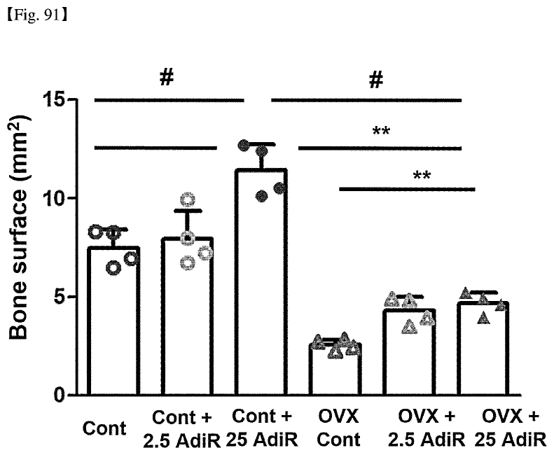

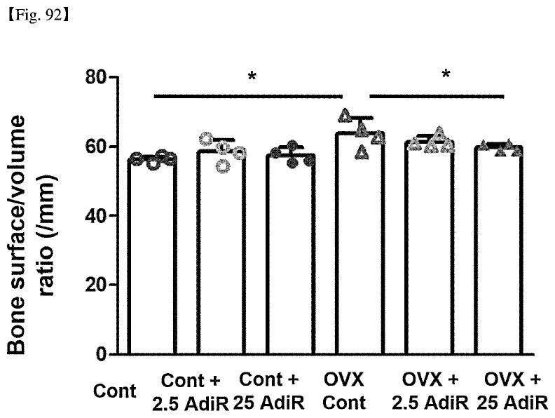

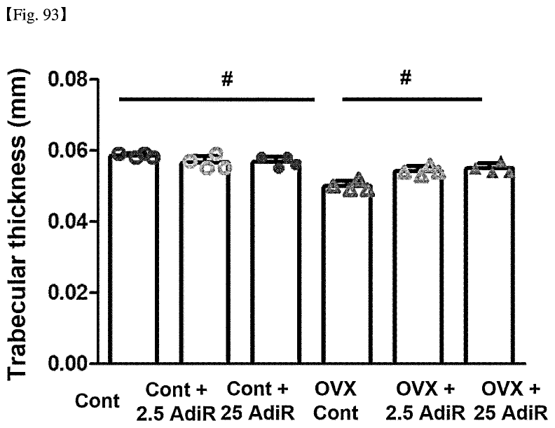

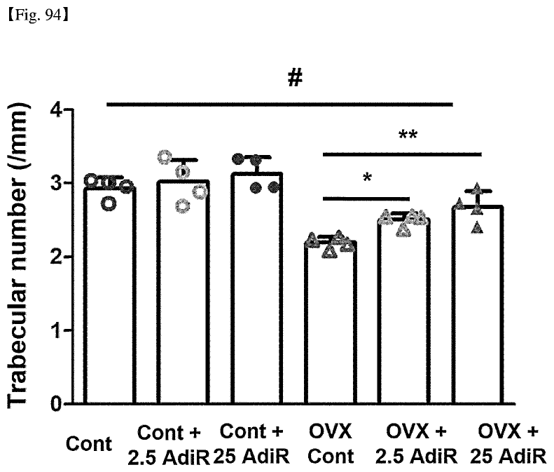

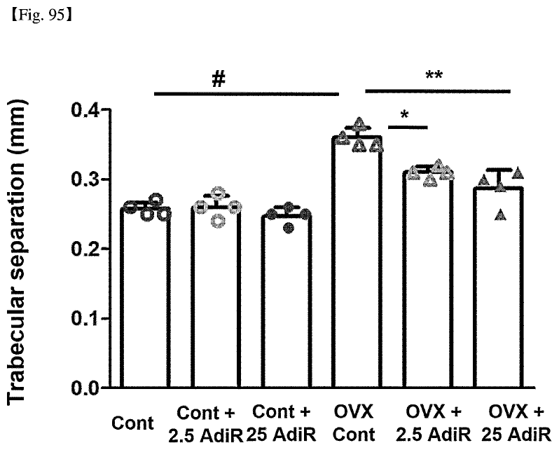

As can be seen from western blot results of adiponectin-receptor 1/2 in ilium (femur) tissue of mice, in ovariectomized mice, both the 2.5 mg/kg day or 25 mg/kg day adiporon-treated groups showed a significant decrease in expression in the ilium tissue of ovariectomized mice not administered with adiporon (P<0.01). This decrease was restored in all the adiporon-administered groups. In addition, it was confirmed that the activation of adiponectin-receptor 1-AMPK, a cell metabolism regulator, and the expression of adiponectin-receptor 2-PPARα and PGC-1α, which were decreased in db/db mice were increased by adiporon administration (FIGS. 80 to 87).

### Effects of Adiporon Treatment on Tissue Changes in Vertebrae (Lumbar Vertebrae 5) in Ovariectomized Mice

From the micro-CT results of the vertebrae (lumbar vertebrae 5) of mice, it was confirmed that the indicators for trabecular thickness, trabecular number, and bone mineral density, bone volume, and bone surface in the vertebrae of ovariectomized mice not administered with adiporon were increased and improved in all of the ovariectomized mouse groups administered with 2.5 mg/kg day or 25 mg/kg day adiporon, respectively (FIGS. 88 to 96).

### Effects of Adiporon Treatment on the Expression of Adiporon Receptor 1/2 and Intracellular Signal Transduction System in Vertebral Bone (Lumbar Vertebrae 5) in Ovariectomized Mice

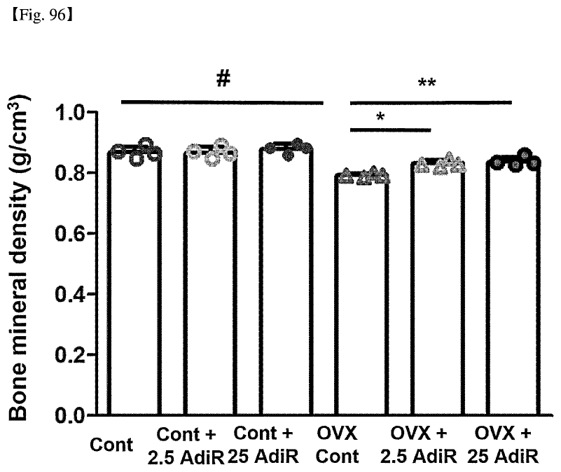

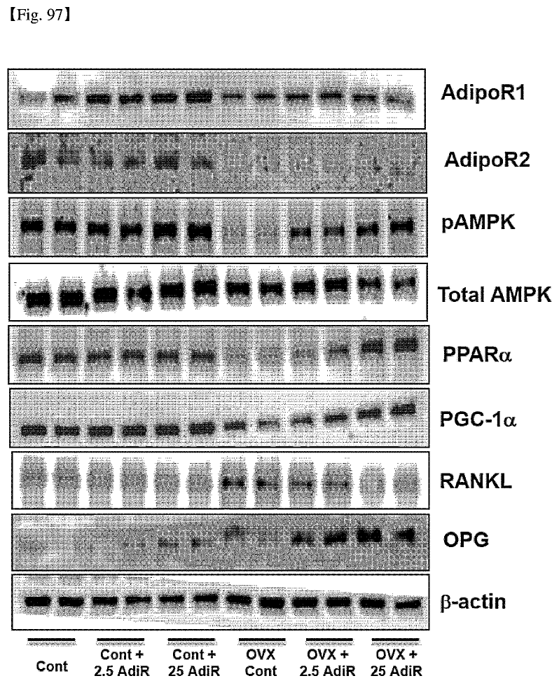

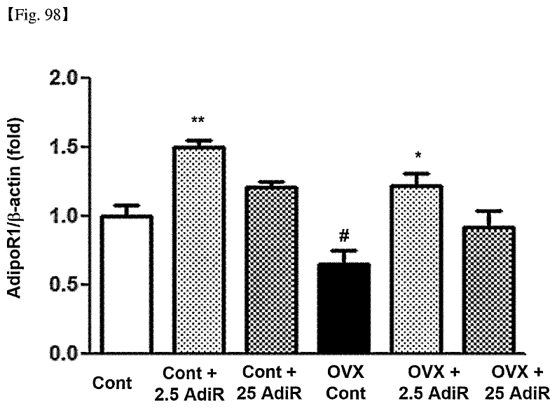

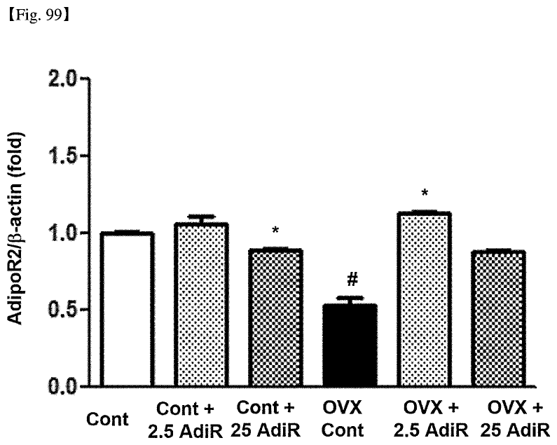

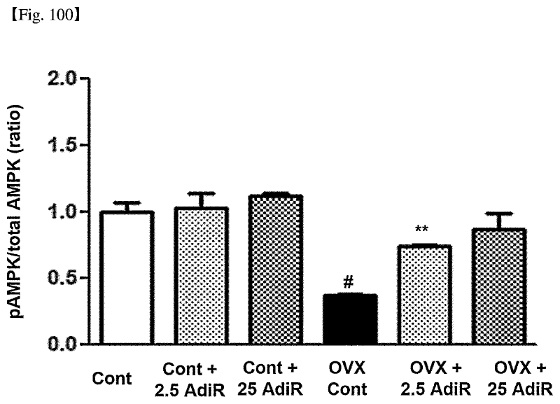

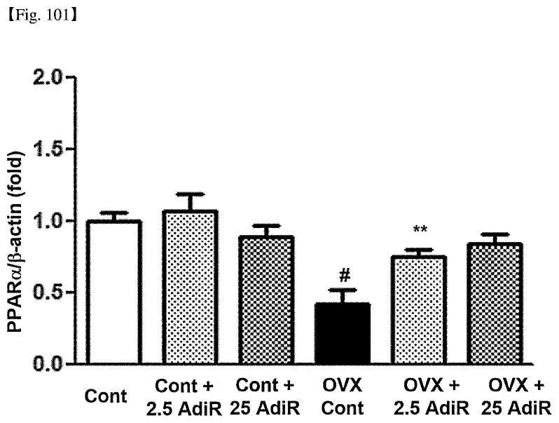

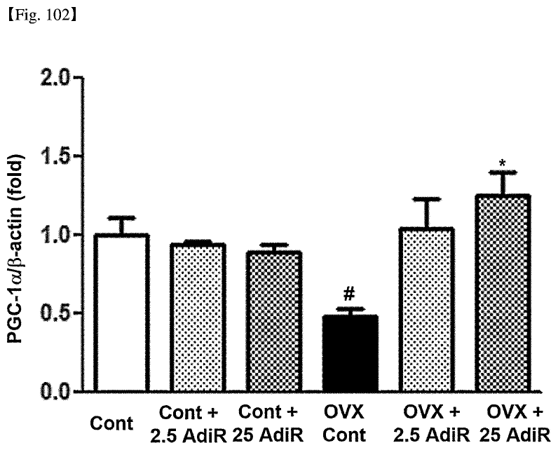

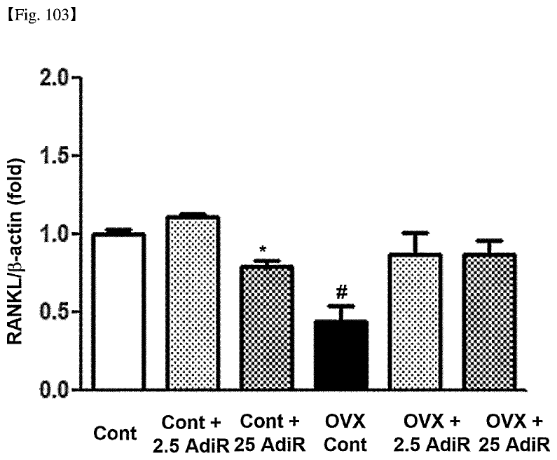

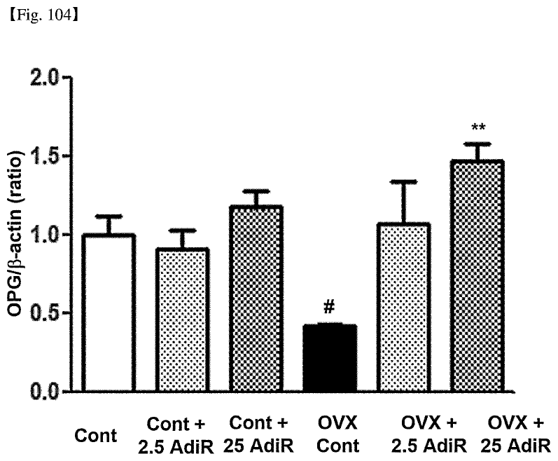

From western blot results of adiponectin-receptor 1/2 in mouse vertebrae (lumbar vertebrae 5) tissue, in ovariectomized mice, both the 2.5 mg/kg day and 25 mg/kg day adiporon-treated groups showed a significant decrease in the expression in the vertebrae tissue of ovariectomized mice not administered with adiporon (P<0.01). Vertebrae of ovariectomized mice not administered with adiponectin in both 2.5 mg/kg day and 25 mg/kg day adiporon-treated groups in ovariectomized mice Expression in tissues showed a significant decrease (P<0.01). This decrease was restored in all of the adiporone-administered groups. In addition, it was confirmed that the activation of adiponectin-receptor 1-AMPK, a cell metabolism regulator, and the expression of adiponectin-receptor 2-PPARα and PGC1α, which were decreased in db/db mice were increased by adiporon administration, (FIGS. 97 to 104).

### Effects of Adiporon on Changes in Serum ACP5/TRAP and Osterocalcin Levels in Ovariectomized Mice

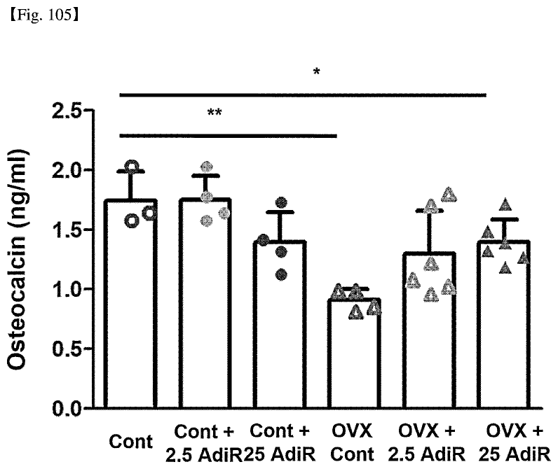

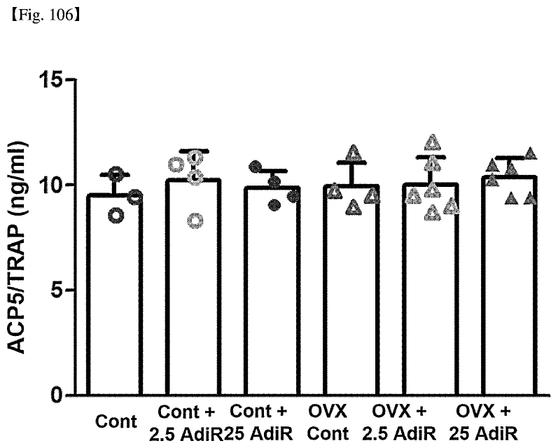

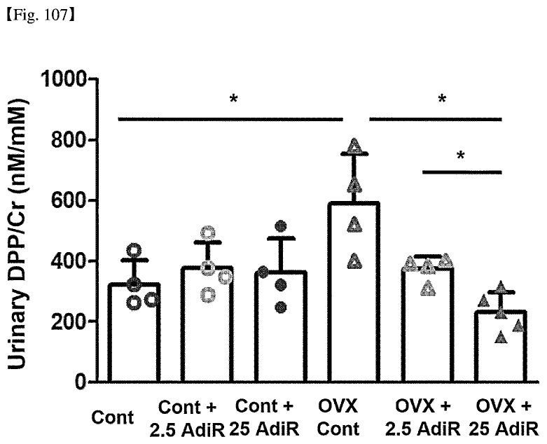

As a result of confirming serum ACP5/TRAP and osteocalcin concentrations by ELISA method, it was confirmed that serum osteocalcin concentration was significantly reduced in ovariectomized mice in both 2.5 mg/kg day and 25 mg/kg day adiporon groups compared to ovariectomized mice not administered adiporon (P<0.01). This decrease was reversed by administration of adiporon (FIGS. 105-107). However, there was no such change in serum ACP5/TRAP concentrations. In addition, the concentration of deoxypyridinoline in urine was significantly increased in ovariectomized mice compared to the Sham surgery control group (P<0.05), and this increase was significantly reduced in both the 2.5 mg/kg. day and 25 mg/kg. day administration groups of adiporon compared to ovariectomized mice not administered with adiporon (P<0.05).

So far, the present invention has been looked at with respect to its preferred embodiments. Those of ordinary skill in the art to which the present invention pertains will understand that the present invention can be implemented in a modified form without departing from the essential characteristics of the present invention. Therefore, the disclosed examples should be considered from an illustrative rather than a limiting point of view. The scope of the present invention is shown in the claims rather than the foregoing description, and all differences within the equivalent range should be construed as being included in the present invention.

1. A pharmaceutical composition for preventing or treating diabetic or postmenopausal osteoporosis, comprising adiporon as an active ingredient.

2. A health functional food for preventing or improving diabetic or postmenopausal osteoporosis, containing adiporon as an active ingredient.

3. A method for treating diabetic or postmenopausal osteoporosis comprising administering a therapeutically effective amount of adiporon to a patient in need thereof.
